(19) United States
(12) Patent Application Publication
(10) Pub. No.: US 2026/0096723 A1
(43) Pub. Date: Apr. 9, 2026

(54) FUNDUS IMAGING SYSTEM WITH AUTOFOCUS

(71) Applicant: Identifeye Health Inc., Guilford, CT (US)

(72) Inventors: Georg Schuele, Portola Valley, CA (US); Carina R. Reisin, Tustin, CA (US); Marco Maetzler, Guilford, CT (US); Brett J. Gyarfas, Aptos, CA (US)

(73) Assignee: Identifeye Health Inc., Guilford, CT (US)

(21) Appl. No.: 19/346,754

(22) Filed: Oct. 1, 2025

Related U.S. Application Data
(60) Provisional application No. 63/704,655, filed on Oct. 8, 2024.

(51) Int. Cl. A61B 3/00; A61B 3/12

(52) U.S. Cl. CPC A61B 3/0075; A61B 3/1225

(57) ABSTRACT

The techniques described herein relate to fundus imaging systems with automatic image capture. An example method for fundus imaging comprises aligning an image path of a fundus camera to an eye pupil of a subject, iteratively executing an autofocus function to determine a plurality of first diopter values, adjusting a focus of a lens of the fundus camera in accordance with a second diopter value, the second diopter value identified from the plurality of first diopter values or determined from the plurality of first diopter values, and capturing, after adjusting the focus of the lens in accordance with the second diopter value, a fundus image of a retina of the subject.

### RELATED APPLICATION

This application claims priority under 35 U.S. C. § 119(e) to U.S. Provisional Application No. 63/704,655, filed Oct. 8, 2024, the content of which is incorporated by reference in its entirety for all purposes.

### FIELD

The techniques described herein relate generally to imaging an eye fundus and, more particularly, to fundus imaging systems with autofocus.

### BACKGROUND

Fundus photography captures an image of an inner lining of a retina of an eye, such as an eye of a human subject, with a fundus camera. The inner lining of the retina may also be referred to as the fundus of the eye. The fundus camera may capture the image by causing the illumination and reflectance of the retina to occur through a common optical path, such as a pupil of the eye. A medical professional may use the image to diagnose, monitor, and/or develop a treatment plan for various ocular and non-ocular diseases.

### SUMMARY OF THE DISCLOSURE

In accordance with the disclosed subject matter, fundus imaging systems with autofocus are provided. Some embodiments relate to a method for fundus imaging. The method comprises aligning an image path of a fundus camera to an eye pupil of a subject, iteratively executing an autofocus function to determine a plurality of first diopter values, adjusting a focus of a lens of the fundus camera in accordance with a second diopter value, the second diopter value identified from the plurality of first diopter values or determined from the plurality of first diopter values, and capturing, after adjusting the focus of the lens in accordance with the second diopter value, a fundus image of a retina of the subject.

Some embodiments relate to an apparatus for fundus imaging. The apparatus comprising at least one memory storing processor-executable instructions, and at least one computer hardware processor configured to execute the processor-executable instructions to perform a method. The method comprising aligning an image path of a fundus camera to an eye pupil of a subject, iteratively executing an autofocus function to determine a plurality of first diopter values, adjusting a focus of a lens of the fundus camera in accordance with a second diopter value, the second diopter value identified from the plurality of first diopter values or determined from the plurality of first diopter values, and capturing, after adjusting the focus of the lens in accordance with the second diopter value, a fundus image of a retina of the subject.

Some embodiments relate to at least one non-transitory computer-readable storage medium comprising processor-executable instructions that, when executed by at least one computer hardware processor, cause the at least one computer hardware processor to perform a method for fundus imaging. The method comprising aligning an image path of a fundus camera to an eye pupil of a subject, iteratively executing an autofocus function to determine a plurality of first diopter values, adjusting a focus of a lens of the fundus camera in accordance with a second diopter value, the second diopter value identified from the plurality of first diopter values or determined from the plurality of first diopter values, and capturing, after adjusting the focus of the lens in accordance with the second diopter value, a fundus image of a retina of the subject.

Some embodiments relate to a fundus imaging system comprising a fundus camera, at least one memory storing processor-executable instructions, and at least one computer hardware processor configured to execute the processor-executable instructions to perform a method for fundus imaging. The method comprising aligning an image path of the fundus camera to an eye pupil of a subject, iteratively executing an autofocus function to determine a plurality of first diopter values, adjusting a focus of a lens of the fundus camera in accordance with a second diopter value, the second diopter value identified from the plurality of first diopter values or determined from the plurality of first diopter values, and capturing, after adjusting the focus of the lens in accordance with the second diopter value, a fundus image of a retina of the subject.

The foregoing summary is not intended to be limiting. Moreover, various aspects of the present disclosure may be implemented alone or in combination with other aspects.

### BRIEF DESCRIPTION OF FIGURES

Various aspects and embodiments will be described with reference to the following figures. In the figures, each identical or nearly identical component that is illustrated in various figures is represented by a like reference character. For purposes of clarity, not every component may be labeled in every drawing. The drawings are not necessarily drawn to scale, with emphasis instead being placed on illustrating various aspects of the techniques and devices described herein.

FIG. 1 is an illustration of an example fundus imaging system including an example fundus camera in communication with an example fundus image computing system and configured to capture an image of a retina of a subject using autofocus, according to some embodiments.

FIG. 2 is a block diagram of an example implementation of the fundus imaging system of FIG. 1, according to some embodiments.

FIG. 3A is a fundus image, according to some embodiments.

FIG. 3B is a fundus image captured with improved autofocus, according to some embodiments.

FIG. 4 is a flowchart representative of an example process that may be performed and/or example machine-readable instructions that may be executed by processor circuitry to implement the fundus imaging system of FIGS. 1 and/or 2 to capture a fundus image of a retina of a subject using autofocus, according to some embodiments.

FIG. 5 is a flowchart representative of an example process that may be performed and/or example machine-readable instructions that may be executed by processor circuitry to implement the fundus imaging system of FIGS. 1 and/or 2 to determine autofocus parameters for capturing a fundus image of a retina of a subject using autofocus, according to some embodiments.

FIG. 6 is an example electronic platform structured to execute the machine-readable instructions of FIGS. 4 and/or 5 to implement the fundus imaging system of FIGS. 1 and/or 2, or portion(s) thereof, according to some embodiments.

### DETAILED DESCRIPTION

The present application generally provides techniques for fundus imaging systems with autofocus. Fundus photography, and/or, more generally, fundus imaging, captures an image and/or photograph of an inner lining of a retina of an eye, such as an eye of a human subject, with a fundus camera. The inner lining of the retina may also be referred to as the fundus of the eye or simply the fundus. The fundus camera may record images of the condition of the fundus to document the presence (or absence) of disorders and monitor their change over time.

A fundus camera (which may also be known as a retinal camera) may be implemented by a specialized, low power microscope with an attached camera designed and/or configured to image the interior surface of the eye, including the retina, retinal vasculature, optic disc, macula, and posterior pole (i.e., the fundus). To facilitate fundus imaging, a human subject's eyes may be dilated before image capture, such as by administering eye drops to each eye to be imaged. Widening (i.e., dilating) a patient's pupil reduces the likelihood of clipping the illumination and detection optical paths while allowing for a larger spatial separation of these two paths, which also assists with mitigating and/or reducing image artifacts such as halo and glare. The increase of imaging angle of observation enables an operator and/or user controlling the fundus camera to image an increased area of the back of the eye and have a larger view thereof. Examples of a fundus camera operator and/or user include a medical doctor (e.g., an ophthalmologist), an optometrist, an optician, and a technician.

Typically, during fundus imaging, a human subject may be instructed to sit in front of the fundus camera with the subject's chin resting on a chin rest (e.g., a fundus camera attachment) and the subject's forehead against a bar or other support structure. The fundus camera user may align and focus the fundus camera on a pupil of a first eye of the subject. For instance, the camera user may move the fundus camera in one or more directions to align an image path to the black center of the subject's eye. The camera user may adjust a lens of the fundus camera to improve focus of the fundus camera (e.g., to reduce blurriness in a fundus image). The adjustment to the lens may be manual or through an automatic process typically referred to as “autofocus”. Once aligned/focused, the user presses the shutter release on the fundus camera, which causes a flash (e.g., a camera flash, an illumination flash) to be fired such that an image and/or photograph of the interior surface of the subject's eye may be captured for subsequent analysis and/or processing. This procedure is repeated to capture an image/photograph of the interior surface of the subject's other eye.

The inventors have recognized several technical challenges with conventional fundus imaging. The user is typically highly trained to overcome the technical challenges to achieve high quality fundus images. The technology developed by the inventors leads to better image quality and also lowers the training requirement of the user so even a novice user can achieve reliable high quality fundus images, or the imaging sequence can be self-administered by the subject.

First, it is challenging for a user to accurately and/or consistently determine when an eye of a human subject is sufficiently opened to facilitate the fundus image capture. For example, a fundus camera may need a pupil to have a width and/or diameter in a range of 3.3 millimeters (mm) to 5 mm to facilitate sufficient fundus capture. With conventional fundus imaging systems, a user may view the subject's pupil, and/or, more generally, the subject's eye, prior to image capture. The user may determine that the subject's eye is not sufficiently opened such that a fundus image of the eye may be captured. The user may instruct the subject to increase the opening of their eye. With the subject's eye being open but not being sufficiently open, the resulting image may be inaccurate such that the intended eye anatomy for capture is not sufficiently present in the captured image. The user may need to take several images until the subject's eye is sufficiently opened to capture the desired image. The several images may thereby expose the subject to extraneous image flashes, which certainly will cause significant pupil constriction and may cause physical discomfort for the subject.

Second, it is challenging for a user to ensure a fundus image is captured without a human subject blinking. The inventors have recognized that images taken of a human subject blinking are just of eyelids and are therefore unusable for fundus imaging. By way of example, the subject may involuntarily close (e.g., partially close, fully close) their eye prior to image capture. With the subject's eye being partially or fully closed, the resulting image is inaccurate because the intended eye anatomy for capture is not present in the captured image. The user may need to take several images until the subject's eye is opened to capture the desired image. The several images may thereby expose the subject to extraneous image flashes, which may cause physical discomfort for the subject.

The inventors have also recognized several technical challenges with focusing conventional fundus imaging systems. For instance, a substantial number (e.g., 20%) of all captured fundus images are unusable because the fundus image is blurry due to a wrong focus adjustment. Out of focus images cause important features of the fundus (e.g., microvessels or micro bleeding) to be indiscernible.

First, the inventors have recognized that fundus cameras that use manual adjustments for focusing a fundus camera lens can produce unusable fundus images due to the varying degree of skill amongst users. For example, some fundus camera users may produce fundus images that are less blurry than other fundus camera users.

Second, the inventors have recognized that fundus cameras that use autofocus adjustments for focusing a fundus camera lens can also produce unusable fundus images. For instance, a fundus camera that captures autofocus images to generate the autofocus function typically utilizes infrared light so the light visibility for the subject is greatly reduced. However, by utilizing infrared light the retinal contrast is reduced, which causes the contrast based autofocus function to not perform reliably. Finding a balance between getting a good image contrast and uncomfortable bright light for the subject is a technical challenge with conventional fundus cameras. Further, selecting a low visibility not to trigger a pupillary constriction, such as in cases when the use of mydriatic agents is to be avoided, is also technically challenging with conventional fundus cameras.

An autofocus function refers to an algorithm and/or technique to detect the correct focus of an image of an object under test, such as an eye of a human subject. Examples of the autofocus function include a fast Fourier transform (FFT) (e.g., a two-dimensional (2-D) FFT), a gradient operator, the Sobel edge detector, and more generally an image contrast based algorithm. Also, more advanced Phase Detection Autofocus (PDAF) as supported by modern image sensors can be utilized. Further, machine learning based techniques for focus determination can be utilized. A fundus camera system that uses autofocus may include an infrared sensor (e.g., a sensor sensitive to infrared light) to image a subject's eye under infrared light exposure. The fundus camera system may use active focusing elements like moveable lenses or active lenses like deformable lenses.

For each focus position, a diopter or position value can be recorded and stored in combination with the output of the image focus algorithm value to form a focus curve. The focus position used to capture a fundus image can be detected by searching for the best focus position of the focus curve. For example, the focus camera can determine (e.g., calculate) an autofocus parameter, such as a diopter value, that corresponds to an ideal focus value of the focus curve.

Typically, a single autofocus function is generated, which may output a diopter value, and then the subject is immediately flashed with the selected diopter value. However, the inventors have recognized that the subject's eye typically moves out of the selected spatial overlap of the fundus camera and the subject's eye due to natural occurring eye motions and limited patient compliance. Further, a typical autofocus function can take approximately 0.5 to 3 seconds, but can also extend up to 9 seconds, which can be far beyond a time in which a subject truly holds still for autofocus and/or fundus image capture. Also image artifacts like motion blur due to eye micro movements like saccades can also misguide the focus algorithm.

Further, the inventors have recognized that a conventional technique for mitigating failed focusing of a fundus camera lens is to take multiple fundus images and select the sharpest fundus image (e.g., the least blurry fundus image). However, the inventors have recognized that this conventional technique is highly undesirable as the subject's compliance is greatly reduced after multiple flashes of the retina. After multiple flashes, the subject becomes uncomfortable, and the likelihood of a usable image is relatively low. Also, the pupil constricts after each flash and with that further reduces the change to take high quality images.

The inventors have developed technology that overcomes the aforementioned technical challenges. The technology developed by the inventors includes a fundus imaging system, which includes a fundus camera configured to capture an image of a human subject's fundus (e.g., a fundus image). In some embodiments, the fundus camera captures a fundus image using autofocus to reduce the blurriness of fundus images such that important features of the fundus (e.g., microvessels) are discernible.

In some embodiments, the fundus imaging system executes (e.g., iteratively executes, sequentially executes) multiple autofocus functions while tracking a subject's pupil, which reduces image artifacts to the autofocus function and ensures that the fundus imaging system understands the uncertainty of the eye specific diopter correction the system provides. In some such embodiments, the fundus camera is aligned (e.g., automatically aligned) to the subject's eye and the alignment follows the subject's pupil to ensure optimal and/or otherwise improved spatial overlap and camera position, which is achieved during the time extension of executing multiple autofocus functions.

In some embodiments, by cycling through multiple autofocus functions, the fundus imaging system can execute a first instance of an autofocus function and determine at least one resulting autofocus parameter value, which can include the diopter value (and/or quality numbers for the sharpness estimator used for the autofocus function and focus fit quality estimates). The fundus imaging system can execute one or more subsequent instances, such as a second instance of the autofocus function, to determine additional autofocus parameter value(s).

In some embodiments, the fundus imaging system can build up statistics of the measured diopter values (and/or the autofocus quality measure(s)) such as by determining a mean, median, and/or mode of the measured diopter values. Any other statistics may be used. A diopter value used to focus (e.g., autofocus) the fundus camera lens can be determined from the mean, median, and/or mode (and/or any other statistic) of the measured diopter values. The fundus imaging system can capture a fundus image of the subject in accordance with the determined diopter value. For example, the fundus imaging system can use one of the measured diopter values or the determined diopter value to focus the fundus camera lens for fundus image capture.

In some embodiments, the fundus imaging system can determine to either continue or stop executing instances of the autofocus function depending on the spread of the measured diopter values (and/or the autofocus quality data). When determining to stop, the fundus imaging system can reoptimize for spatial pupil overlap by realigning the fundus camera to the subject's pupil. The fundus imaging system can capture a fundus image of the subject in accordance with the determined diopter value after fundus camera is realigned to the subject's pupil.

In some embodiments, the fundus imaging system performs blink detection to determine whether a human subject blinked during fundus imaging. For example, the fundus imaging system can avoid triggering a fundus image capture when the subject is blinking. In such an example, the fundus imaging system can detect the presence or absence of a pupil. The fundus imaging system might also specifically ask the subject (e.g., via an output device such as a speaker) to blink or wait for a blink to naturally occur and flash the eye right after the blink as this minimizes the chance that the subject is blinking again. For example, the fundus imaging system can determine whether the subject blinked and capture the fundus image after determining that an eyelid of the subject reopened after blinking.

In some embodiments, the fundus imaging system can also determine, after a fundus image is taken, whether it is a proper fundus image. For example, the fundus imaging system can determine that the image has artifacts, and determine that the image should be retaken (e.g., automatically retaken) when the pupil reaches a sufficient pupil size again. Beneficially, these techniques developed by the inventors will ensure that an optimal fundus image is taken with minimal burden for the subject.

The techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

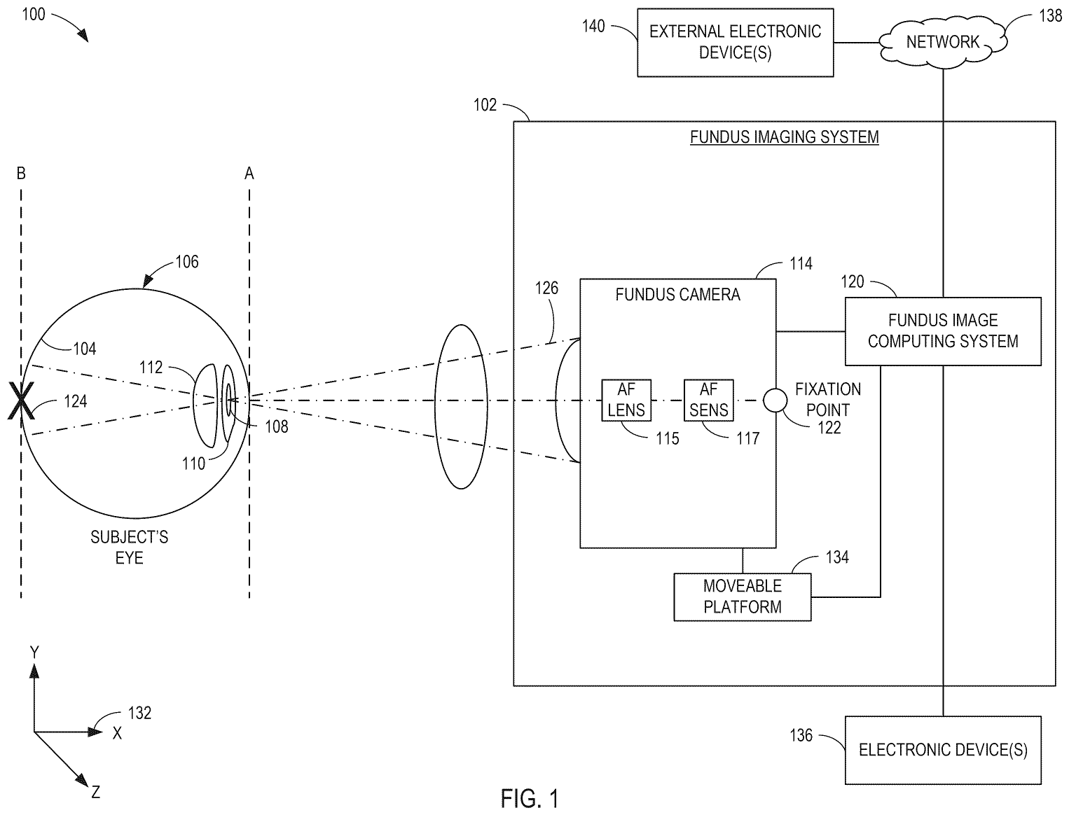

Turning to the figures, the illustrated example of FIG. 1 is an illustration of an example fundus imaging environment 100 including an example fundus imaging system 102 that can be configured to capture an image of a retina 104 of an eye 106 of a subject through a pupil 108 of the eye 106. Also depicted is an iris 110 and a lens 112 of the eye 106. Other eye anatomical objects, such as the sclera, choroid, and cornea are not shown for enhanced clarity, but nonetheless may be present. Although only one eye 106 is depicted, it should be understood that the fundus imaging system 102 can be configured to capture an image of multiple retinas, such as the retina 104 of the eye 106, which is shown, and another retina of a different eye of the subject, which is not shown. For example, the fundus imaging system 102 can be configured to capture a first fundus image of the retina 104 and a second fundus image of the other retina substantially simultaneously.

The term “substantially simultaneously” may refer to occurrence in a near instantaneous manner recognizing there may be real-world delays for computing time, transmission, etc. For example, the fundus imaging system 102 can be configured to capture the first and second fundus images within 30 seconds, 1 second, 500 milliseconds, 100 milliseconds, 10 milliseconds, etc., of real time of each other.

The eye 106 of the subject of this example is a human eye of a human subject. Examples of human subjects include infants, children, adolescents, adults, and older adults (e.g., adults 65 years of age or older). Alternatively, the eye 106 may be an animal eye of an animal subject. Examples of animal subjects include mice, cats, dogs, primates, and horses.

The fundus imaging system 102 of the illustrated example includes a fundus camera 114 that can be configured to capture an image, such as a fundus image, of the retina 104. For example, the fundus camera 114 can be a retinal camera configured to capture an image of the retina 104. The fundus camera 114 of this example implements a visualization system configured to generate and/or output a fundus image. For example, the fundus camera 114 and/or, more generally, the visualization system, can be implemented by a specialized, low power microscope with an attached camera designed and/or configured to image the interior surface of the eye 106, including the retina 104, retinal vasculature, optic disc, macula, and posterior pole (i.e., the fundus).

The fundus camera 114 of the illustrated example includes an autofocus lens 115 (identified by AF LENS). In some embodiments, the autofocus lens 115 is a lens that can be adjusted to change a focus of a fundus image captured by the fundus camera 114. For example, the autofocus lens 115 can be adjusted by being actuated to move closer to the subject's eye 106 or away from the subject's eye 106. In such an example, the autofocus lens 115 can include and/or be coupled to one or more actuators to move the autofocus lens 115 towards or away from the subject's eye 106.

In some embodiments, the autofocus lens 115 is a set of lenses that can be adjusted to change a focus of a fundus image captured by the fundus camera 114. For example, the set of lenses can be adjusted by actuating one or more lenses of the set of lenses to change the distance between themselves. For example, a lens from the set of lenses can be actuated to move closer to the subject's eye 106 or away from the subject's eye 106. In such an example, the autofocus lens 115 can include and/or be coupled to one or more actuators to move one or more lenses of the set of lenses towards or away from the subject's eye 106.

In some embodiments, the autofocus lens 115 can be actuated (e.g., automatically actuated) in response to commands from a fundus image computing system 120. Additionally or alternatively, the autofocus lens 115 may be actuated (e.g., manually actuated) by a user (e.g., a human user) of the fundus imaging system 102.

In some embodiments, a user of the fundus imaging system 102 is a human user. In some embodiments, a user of the fundus imaging system 102 is a machine user such that the fundus imaging system 102 controls itself via automatic operation.

In some embodiments, shape changing autofocus lenses like a liquid lens can be implemented. For example, the autofocus lens 115 can be implemented by one or more shape changing autofocus lenses (e.g., one or more liquid lenses).

The fundus camera 114 of the illustrated example includes an autofocus sensor 117 (identified by AF SENS). The autofocus sensor 117 is a sensor configured to output a measurement that can be used to determine adjustment(s) to the autofocus lens 115.

Alternatively, the autofocus sensor 117 may include more than one sensor and/or more than one type of sensor.

In some embodiments, the autofocus sensor 117 can include and/or be coupled to one or more actuators to move the autofocus sensor 117 towards or away from the subject's eye 106. In some embodiments, the autofocus sensor 117 can be actuated (e.g., automatically actuated) in response to commands from the fundus image computing system 120. Additionally or alternatively, the autofocus sensor 117 may be actuated (e.g., manually actuated) by a user of the fundus imaging system 102. In some embodiments, the autofocus lens 115 and the autofocus sensor 117 can be actuated by the same one or more actuators such that the autofocus lens 115 and the autofocus sensor 117 can be moved together.

In some embodiments, the autofocus sensor 117 itself can be actuated and used for focus adjustment. In such an example the autofocus sensor position is optimized to place the autofocus sensor 117 in the perfect and/or optimized optical relay point to a target eye location 124. The target eye location 124 of this example is the retina. In this scenario, the autofocus sensor 117 also is used as the image sensor for the actual fundus image. There may also be a small offset between the infrared autofocus position and the visible light image focus position due to chromatic aberrations of the optical system between the infrared and visible wavelength.

In some embodiments, the autofocus sensor 117 includes and/or is implemented by an infrared emitter and an infrared detector. Alternatively, the autofocus sensor 117 may include and/or be implemented by a visible light emitter and a visible light detector. The infrared emitter and the infrared detector may collectively implement an infrared sensor. Alternatively, the infrared detector may implement an infrared sensor. The infrared emitter can be configured to emit infrared light towards the subject's eye 106, which bounces back to the infrared detector. The infrared detector can process the measured infrared reflection into an output representative of a distance between the infrared emitter and the subject's eye 106, or portion thereof. The fundus image computing system 120 may receive the output and actuate the autofocus lens 115 to move closer towards or further away from the subject's eye 106 based on the distance between the infrared emitter and the subject's eye as indicated by the output.

An example of an infrared emitter is a light emitting diode (LED) (e.g., an infrared LED), but other types of infrared emitters may be used. An example of the infrared detector is a photodiode (e.g., an infrared photodiode) and a charge coupled device (CCD), or a complementary metal-oxide-semiconductor (CMOS) sensor, but other types of infrared detectors may be used such as infrared photoconductive detectors, pyroelectric detectors, and bolometers (e.g., microbolometers).

A CCD/CMOS sensor can be implemented by a light-sensitive integrated circuit that captures images by converting photons to electrons. In such an example, the surface of the CCD can include a plurality of pixels (e.g., thousands of pixels, tens of thousands of pixels) to convert granular incidents of light (e.g., infrared light, visible light) onto the plurality of pixels into electrical energy, which can be organized into digital data (e.g., a digital image). In some embodiments, the digital image is an autofocus image, which refers to an image that the fundus image computing system 120 may process to focus the autofocus lens 115. Alternatively, the autofocus sensor 117 may be a CMOS sensor.

The fundus image computing system 120 may receive the output from the CCD (e.g., the digital data). The fundus image computing system 120 may process the output to determine changes in contrast between subsequent digital images of the subject's eye. The fundus image computing system 120 may actuate the autofocus lens 115 (and/or the autofocus sensor 117) to move closer towards or further away from the subject's eye 106 based on the determined contrast between the subsequent digital images. For example, the fundus image computing system 120 may actuate the autofocus lens 115 to move to improve the contrast detected in a subsequent digital image of the subject's eye and thereby improve the focus of the autofocus lens 115.

In the illustrated example, the fundus imaging system 102 includes the fundus image computing system 120, which can be configured to analyze, evaluate, and/or process outputs from at least one of the fundus camera 114, the autofocus lens 115, or the autofocus sensor 117. The fundus image computing system 120 of this example is coupled (e.g., communicatively coupled, electrically coupled, physically coupled) to the fundus camera 114 and/or portion(s) thereof, such as the autofocus lens 115 and/or the autofocus sensor 117. Alternatively, the fundus image computing system 120 may be separate from and/or otherwise not be included in the fundus imaging system 102. In some embodiments, the fundus image computing system 120 may also correct the focus position for chromatic aberrations of the optical system between the infrared autofocus light and the visible light used to image the retina.

In some embodiments, the subject may be undergoing an eye exam such that eye drops are administered to the eye cornea (not shown) to cause their pupils to dilate. The subject may be instructed to sit in front of the fundus camera 114 with the subject's face resting in a shape matching goggle interface or a chin resting on a chin rest (not shown) and the subject's forehead against a bar or other support structure (not shown). The subject may be instructed to fixate and/or otherwise look at a fixation point 122. The fixation point 122 may be a fixation target implemented by a light projector (not shown). In some embodiments, the fixation point 122 may be a dot or other pattern implemented by light, such as a light emitted by one or more LEDs.

In some embodiments, the subject may be instructed to gaze towards the fixation point 122 during the eye exam such that a target eye location 124 (identified by an “X” on the retina 104) of the eye 106 can be recorded and/or captured. For example, the fundus camera 114 can be triggered to capture a fundus image of the target eye location 124 by leveraging a first imaging path 126 of the fundus camera 114 that traverses into and out of the pupil 108. The target eye location 124 of this example is the macula of the eye 106. The macula of eye 106 is the round area at the center of the retina 104. Alternatively, any other target eye location is contemplated such as the optic discs (not shown).

In some embodiments, the fundus imaging system 102 can align (e.g., automatically align) the first imaging path 126 of the fundus camera 114 with the pupil 108 such that the target eye location 124 can be recorded and/or captured. In some embodiments, the fundus image computing system 120 can determine that the pupil 108 is not detected in a fundus image and/or is not aligned (e.g., approximately aligned within 1 degree, 2 degrees, 5 degrees, etc., of a major and/or minor axis) in the fundus image. In some embodiments, the fundus image computing system 120 can determine to move the fundus camera 114 such that the pupil 108 is detected in a fundus image and/or is aligned in the fundus image. For example, the fundus image computing system 120 can generate and/or output control signal(s) representative of command(s) to a moveable platform 134 to move the fundus camera 114 along one or more directions (e.g., an x-direction, a y-direction, a z-direction) of a camera-based coordinate system 132. an x-direction, a y-direction, and/or a z-direction of a camera. The camera-based coordinate system 132 of this example defines points relative to the axial center of the fundus camera 114. Alternatively, the fundus imaging environment 100 may utilize a calibration pattern-based coordinate system in which points are defined relative to a point in the scene.

In some embodiments, the moveable platform 134 may be implemented by one or more stages, one or more actuators, and/or one or more position sensors. The stage(s) can be configured to support, hold, and/or move an object, such as the fundus camera 114. Examples of the stage(s) include one or more boards, planks, or platforms that is/are coupled to the fundus camera 114.

The actuator(s) of the moveable platform 134 can be configured to move and/or translate the stage(s) in one or more directions. In some embodiments, the actuator(s) is/are motors. Examples of motors include alternating current (AC) brush motors, AC brushless motors, direct current (DC) brush motors, DC brushless motors, stepper motors, servo motors, linear motors, and direct drive motors.

By way of example, the actuator(s) can be operatively coupled to the stage(s), which can be coupled to the fundus camera 114. For example, the actuator(s) can include a plurality of actuators including one or more first actuators, one or more second actuators, and/or one or more third actuators. In some embodiments, the one or more first actuators of the actuator(s) can be operatively coupled to a first stage of the stage(s) such that the one or more first actuators can be actuated and/or otherwise controlled to move the first stage and thereby the fundus camera 114 in a first direction, such as the x-direction of the camera-based coordinate system 132. In some embodiments, the one or more second actuators of the actuator(s) can be operatively coupled to a second stage of the stage(s) such that the one or more second actuators can be actuated and/or otherwise controlled to move the second stage and thereby the fundus camera 114 in a second direction, such as the y-direction of the camera-based coordinate system 132. In some such embodiments, the y-direction is orthogonal to the x-direction. In some embodiments, the one or more third actuators of the actuator(s) can be operatively coupled to a third stage of the stage(s) such that the one or more third actuators can be actuated and/or otherwise controlled to move the third stage and thereby the fundus camera 114 in a third direction, such as the z-direction of the camera-based coordinate system 132. In some such embodiments, the z-direction is orthogonal to the x-direction and the y-direction.

The position sensor(s) of the moveable platform 134 can be configured to detect and/or determine a position of respective one(s) of the stage(s). Examples of the position sensor(s) include capacitive displacement sensors, eddy current sensors, Hall effect sensors, inductive sensors, linear variable differential transformers, piezo-electric transducers, position encoders (e.g., absolute encoders, incremental encoders, linear encoders, rotary encoders), potentiometers, proximity sensors, and ultrasonic sensors. By way of example, the position sensor(s) can include one or more of a plurality of position sensors. In some embodiments, the plurality of position sensors can include a first position sensor configured to determine a position of the first stage of the stage(s), a second position sensor configured to determine a position of the second stage of the stage(s), and/or a third position sensor configured to determine a position of the third stage of the stage(s).

In some embodiments, the fundus camera 114 executes multiple autofocus functions while tracking the pupil 108, which beneficially improves the reliability of the autofocus functions. By way of example, the fundus camera 114 via the moveable platform 134 can be aligned (e.g., automatically aligned) to the subject's eye 106 to ensure optimal and/or otherwise improved spatial overlap and camera position, which is achieved during the time extension of running multiple autofocus functions.

Furthering the example, the fundus camera 114 can cycle and/or iterate through multiple autofocus functions. In some embodiments, the fundus camera 114 cycles/iterates through different instances of the same autofocus function while continuing to track the pupil. Additionally or alternatively, the fundus camera 114 may cycle/iterate through different types of autofocus functions.

Continuing the example, the fundus camera 114 can effectuate an autofocus function by outputting an autofocus image to the fundus image computing system 120 at different positions along the x-axis in a range of positions. In some embodiments, each of the different positions can correspond to a respective diopter value in a range of diopter values. An example diopter (D) value range is −20 D to +20 D. Alternative ranges may be used such as 15 D to +10 D. For example, the fundus camera 114 can capture an autofocus image at −20 D, another autofocus range of −19 D, and so on. Although a step size of 1 is provided, other step sizes are contemplated such as 0.1, 0.2, 0.3, 0.4, 0.5, etc.

In some embodiments, the fundus camera 114 may use a refractometer, such that a diopter value can be determined using fundus images. In some embodiments, the fundus camera 114 may determine a diopter value using optical coherence tomography (OCT).

At each position, the fundus camera 114 can emit infrared light, via the autofocus sensor 117, which is bounced back onto an infrared detector, such as a CCD. The CCD can output digital data representative of an autofocus image to the fundus image computing system 120. The fundus image computing system 120 can control the autofocus lens 115 and the autofocus sensor 117 to move to the next position after receiving the autofocus image.

The fundus image computing system 120 can execute an autofocus function by processing the autofocus image, using an FFT, to output a focus value at each position along the x-direction. For example, the autofocus function can take the current input autofocus image and a height and width of the current input autofocus image and output a focus value for each position with a step size, which can depend on the selected autofocus function.

In some embodiments, the focus value can be determined based on a number of identified high-frequency components (e.g., frequency data, high-frequency data), such as image data associated with microvessels in the subject's eye 106 or other high contrast edges, in the autofocus image. For example, the fundus image computing system 120 can convert the autofocus image into frequency data, which may include high-frequency components and/or low-frequency components. The fundus image computing system 120 can identify a candidate diopter value for lens focusing based on which autofocus image of one or more autofocus images has the greatest number of the high-frequency components.

By way of example, the focus value can increase with an increasing number of identified high-frequency components and decrease with a decreasing number of identified high-frequency components. In such an example, a first autofocus image with a first number of high-frequency components can have a first focus value greater than a second focus value of a second autofocus image with a fewer number of high-frequency components than the first autofocus image. In this way, the fundus image computing system 120 can determine differences in contrast between autofocus images and determine, using the focus values, which of the autofocus images have the sharpest contrast and thereby have the best focus. Also phase detection autofocus information can be used to determine optimal focus position or a combination of phase and contrast based system.

The fundus image computing system 120 can determine, using the focus values along the x-direction, a focus curve. The focus curve can include a plurality of focus values. The fundus image computing system 120 can determine a maximum focus value of the focus curve. The fundus image computing system 120 can determine a diopter value of the autofocus lens 115 that corresponds to the maximum focus value.

In some embodiments, the fundus camera 114 can execute another autofocus function, such as another instance of the same autofocus function described above or a different type of autofocus function. For example, the fundus camera 114 can control the autofocus sensor 117 to move along the range of positions in the x-direction and outputting focus values at each respective position. The fundus camera 114 can output another focus curve and determine another diopter value that corresponds to the maximum focus value in the focus curve.

In some embodiments, the fundus camera 114 can execute a plurality of autofocus functions (e.g., a plurality of instances of the same autofocus function, a plurality of different autofocus functions) to output a plurality of diopter values. The fundus camera 114 can process the plurality of diopter values into an autofocus parameter, which can correspond to an optimal and/or otherwise improved diopter value to configure the autofocus lens 115 for fundus image capture. For example, the fundus camera 114 can determine an autofocus parameter by determining a mean (e.g., an average), median, or mode of the plurality of diopter values. Alternatively, the fundus camera 114 may determine the autofocus parameter by utilizing a larger ensemble fit of all the diopter values to determine an optimized and/or otherwise improved diopter value. In such an example, the autofocus parameter can be a diopter value that is a mean of the plurality of diopter values. In another example, the autofocus parameter can be a diopter value that is a median of the plurality of diopter values. In yet another example, the autofocus parameter can be a diopter value that is a mode of the plurality of diopter values. Additionally and/or alternatively, machine learning based techniques can be utilized to determine the best focus position.

In some embodiments, the fundus camera 114 determines to stop executing autofocus functions. For example, the fundus camera 114 can determine to cease autofocus function execution depending on the spread of the resulting diopter values. Alternatively, the fundus camera 114 may determine to continue executing autofocus functions based on the spread of the resulting diopter values.

After the fundus camera 114 determines to stop executing autofocus functions, the fundus camera 114 may realign itself to the pupil 108 for improved and/or otherwise enhanced spatial pupil overlap. In some embodiments, the fundus camera 114 may add one or more seconds between the ceasing of the autofocus functions and the taking of the fundus image to allow the pupil 108 to dilate as some of the autofocus infrared light may still be visible and a pupillary dilation is still feasible.

In some embodiments, the subject may be asked to blink before taking a fundus image. For example, a user (e.g., a human user, a machine user such as the fundus image system 102) may ask the subject to blink. In such an example, a GUI on the electronic device(s) 136 may prompt the human user to ask the subject to blink. In another example, the electronic device(s) 136 may output audio representative of an audible command to the subject to blink before the fundus camera 114 takes a fundus image.

In some embodiments, the fundus camera 114 can configure the autofocus lens 115 in accordance with the autofocus parameter. For example, the fundus camera 114 can adjust the autofocus lens 115 in accordance with a diopter value, which is obtained by taking an average or other statistical processing of the plurality of diopter values. After configuring the autofocus lens 115, the fundus camera 114 can capture a fundus image using the optimal and/or otherwise improved configuration of the autofocus lens 115.

In the illustrated example, the fundus camera 114 can capture an image, such as a fundus image, of the target eye location 124 in accordance with a dynamic pupil trigger associated with the subject undergoing fundus imaging. Typically, when taking the images of a patient's fundus, the patient looks into a fundus camera, then a flash occurs in order to get a well-lit image of the fundus. The image quality improves with bigger pupil size, as light does not get blocked by the pupil and more light enters the eye to better illuminate the fundus (back of the eye) during the imaging. Advantageously, the fundus camera 114, and/or, more generally, the fundus imaging system 102, monitors pupil size during the examination and captures fundus images when the pupil is detected to be at its largest.

The pupil size adapts itself as a response to external light. For example, the pupil gets bigger when light is dimmed and gets smaller when light is bright to regulate the amount of light that gets into the eye. Typically, before the first fundus image is taken, the pupil adapts to the room and/or device dimmed light setting and gets bigger. After an image is taken by flashing the eye with light, the pupil first contracts as a reaction to the flash and then expands back based on the room light. Different people adapt differently to the light environment and the time that it gets to expand back to the dimmed room light. The inventors have recognized that, while images can be triggered based on timer and waiting period from the patient entering the room and time in between images, this is very inconvenient for the patient and does not mean that the pupil will be at its biggest size. The inventors have appreciated that, to minimize examination time and still take images when pupil size is the biggest and focus is maximized, pupil size can be monitored (e.g., monitored continuously) and focus can be adjusted (e.g., adjusted continuously) and trigger exposure/image based pupil size and/or focus. Thus, the fundus camera 114 and/or, more generally, the fundus imaging system 102, can trigger fundus image capture based on the pupil size reaching a threshold pupil size, finding a diopter value corresponding to an optimal focus of the autofocus lens 115, learning from first and/or subsequent image(s) to improve timing of the trigger of the next image (e.g., within some range of pupil size when the previous image was taken), and/or any combination(s) thereof with or without a timing component.

In the illustrated example, the fundus camera 114 can capture an image, such as a fundus image, of the target eye location 124 by performing blink detection. For example, the fundus image computing system 120 may first detect the pupil 108 prior to measuring its size. In such an example, the fundus image computing system 120 may detect the pupil 108 by looking for a black circle in the field of view of the fundus camera 114. When a patient blinks, the pupil 108, and therefore the black circle, will go away. If a patient is blinking, the fundus image computing system 120, which may execute and/or perform a pupil tracking algorithm, can instruct the fundus camera 114 not to capture the picture at that moment until the pupil 108 reappears and is back to the proper size. Additionally or alternatively, if a picture is captured, the fundus image computing system 120 may determine whether the picture is a proper fundus image. If the image is blank, such as an image of an eyelid because the image was taken during the patient's blinking, the fundus image computing system 120 may instruct the fundus camera 114 to take (e.g., automatically take) another image when the pupil 108 hits its maximum size again.

Beneficially, the fundus camera 114 overcomes the technical challenges of focusing conventional fundus imaging systems by capturing a fundus image in accordance with a diopter value determined from a plurality of autofocus functions. For example, by executing multiple autofocus functions, the fundus camera 114 can determine a diopter value that provides for optimal and/or otherwise improved sharpness of an eventual fundus image with reduced and/or otherwise minimized fundus camera user intervention. Beneficially, in some embodiments, by taking a single fundus image using an optimal diopter value instead of taking multiple fundus images using a non-optimal diopter value, the likelihood of capturing a usable fundus image is increased while also reducing subject discomfort from undergoing multiple image flashes. Further, and beneficially, by capturing a fundus image when the pupil 108 meets and/or exceeds a pupil size threshold, the fundus camera 114, and/or, more generally, the fundus imaging system 102, can capture the fundus image with improved accuracy and/or efficiency compared to conventional fundus photography techniques.

In some embodiments, the fundus image can be displayed on a graphical user interface (GUI) implemented by electronic device(s) 136. Examples of electronic devices include laptop computers, tablet computers, cellular phones (e.g., smartphones), televisions (e.g., smart televisions), and wearable devices (e.g., headsets, smartwatches, smart glasses, etc.). For example, the fundus image computing system 120 can output the fundus image to the electronic device(s) 136 via one or more interfaces. Examples of interfaces include BLUETOOTH®, an Ethernet interface, a near-field communication (NFC) interface, a Universal Serial Bus (USB) interface (e.g., USB Type-A, USB Type-B, USB TYPE-C™ or USB-C™, etc.), etc., and/or any combination(s) thereof. The electronic device(s) 136 can generate and/or launch a GUI to present the fundus image, and/or information associated thereof, to the user of the fundus imaging system 102. Alternatively, the electronic device(s) 136 may be part of the fundus imaging system 102.

In some embodiments, the fundus image computing system 120 may output and/or cause transmission of fundus images to external electronic device(s) 140 via at least one network 138. Examples of the external electronic device(s) 140 include computer servers, laptop computers, tablet computers, and cellular phones (e.g., smartphones). In some embodiments, the external electronic device(s) 140 may be implemented at least in part by a cloud computing network (e.g., a private cloud, a public cloud).

The network 138 of this example may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more wireless local area networks (WLANs), etc., and/or any combination(s) thereof. For example, the network 138 may be the Internet, but any other type of private and/or public network is contemplated.

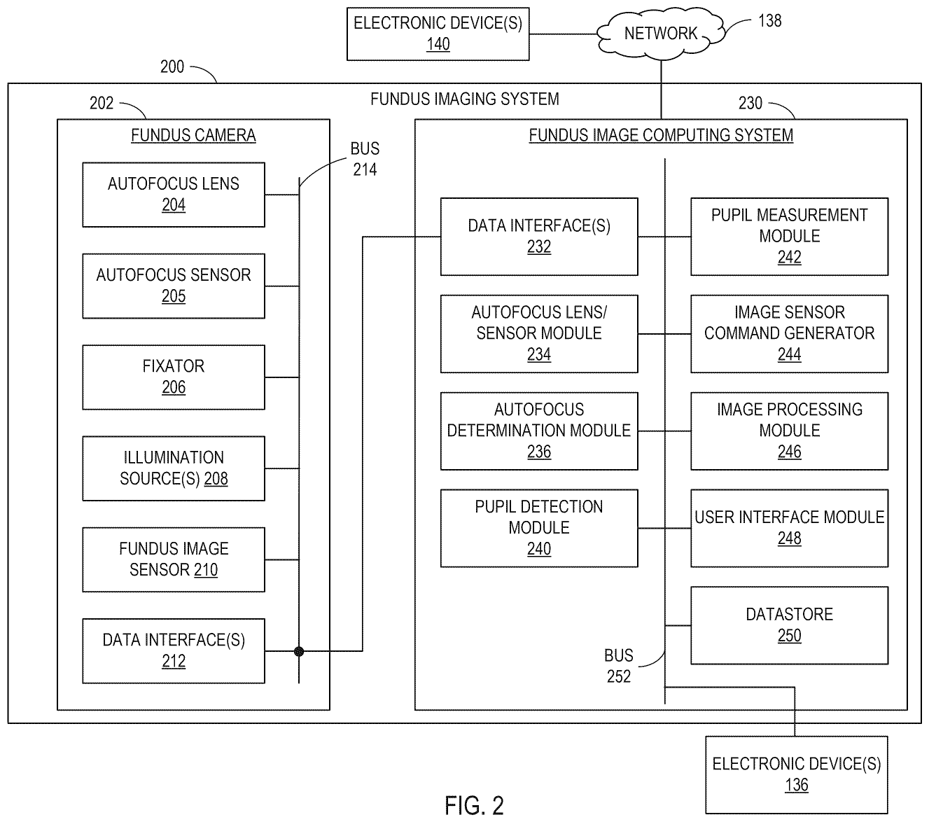

FIG. 2 is a block diagram of an example implementation of a fundus imaging system 200, which includes example implementations of a fundus camera 202 and a fundus image computing system 230. In some embodiments, the fundus imaging system 200 of FIG. 2 can implement and/or correspond to the fundus imaging system 102 of FIG. 1. In some embodiments, the fundus camera 202 of FIG. 2 can implement and/or correspond to the fundus camera 114 of FIG. 1. In some embodiments, the fundus image computing system 230 of FIG. 2 can implement and/or correspond to the fundus image computing system 120 of FIG. 1. The fundus camera 202 of the illustrated example includes an autofocus lens 204, an autofocus sensor 205, a fixator 206, illumination source(s) 208, a fundus image sensor 210, and first data interface(s) 212.

Component(s) of the fundus camera 202 of this example can be in communication with one(s) of each other via a first bus 214. Examples of the first bus 214 can be an Inter-Integrated Circuit (I2C) bus, a Peripheral Component Interconnect (PCI) bus, a Peripheral Component Interconnect Express (PCIe) bus, and a Serial Peripheral Interface (SPI) bus. Any other type of bus, such as a computing and/or electrical bus, is contemplated.

The autofocus lens 204 of the illustrated example can be configured to change a focus of a fundus image. For example, the autofocus lens 204 can be configured to move along the x-direction of the camera-based coordinate system 132 of FIG. 1 to improve the focus of the fundus image captured by the fundus camera 114. In some embodiments, the autofocus lens 204 can correspond to and/or be implemented by the autofocus lens 115 of FIG. 1.

The autofocus sensor 205 of the illustrated example can be configured to output a measurement that can be used to determine adjustment(s) to the autofocus lens 115. In some embodiments, the measurement is representative of a distance between the autofocus sensor 205 and a portion of the subject's eye 106, such as the target eye location 124 of FIG. 1. In some embodiments, the autofocus sensor 205 is a single sensor or includes two or more sensors and/or components.

In some embodiments, the autofocus sensor 205 is implemented by an emitter (e.g., an infrared emitter) and a detector (e.g., an infrared detector). In some embodiments, the emitter/detector can be used to emit light, such as infrared, and output digital data after detecting the emitted light after being bounced back towards the detector. The digital data can be representative of a digital image and/or converted into a digital image. In some embodiments, the autofocus sensor 205 can correspond to and/or be implemented by the autofocus sensor 117 of FIG. 1.

The fixator 206 of the illustrated example can be configured to generate a fixation point, such as the fixation point 122 of FIG. 1. For example, the fixator 206 can generate a dot or other pattern implemented by light. Examples of the fixator 206 include a visible light source and one or more LEDs.

The illumination source(s) 208 of the illustrated example can be configured to emit light momentarily to effectuate capture of an image. For example, the illumination source(s) 208 can be a light source that implements a camera and/or photography flash. Examples of the illumination source(s) 208 include transparent glass tubes filled with an inert gas (e.g., xenon gas or other noble gas), a quartz tube filled with an inert gas, and an LED.

The fundus image sensor 210 of the illustrated example can be configured to generate and/or output a fundus image, such as an image of the target eye location 124 of FIG. 1. In some embodiments, the fundus image sensor 210 can implement and/or correspond to image sensor(s) of the fundus camera 114 of FIG. 1. In some embodiments, the fundus image sensor 210 can include one or a plurality of image sensors. Examples of the fundus image sensor 210 include a CCD device, a CMOS sensor, and an infrared sensor.

The first data interface(s) 212 of the illustrated example can be configured to transmit data to and/or receive data from the fundus image computing system 230. For example, the first data interface(s) 212 can be a bus. In some embodiments, the first data interface(s) 212 can be one or more communication interfaces.

In the illustrated example of FIG. 2, the fundus imaging system 200 includes the fundus image computing system 230 to analyze, evaluate, and/or process data, such as image data, captured and/or generated by the fundus camera 202. The fundus image computing system 230 of the illustrated example includes second data interface(s) 232, an autofocus lens and/or sensor module 234 (identified by autofocus lens/sensor module 234), an autofocus determination module 236, a pupil detection module 240, a pupil measurement module 242, an image sensor command generator 244, an image processing module 246, a user interface module 248, and a datastore 250.

Component(s) of the fundus image computing system 230 of this example can be in communication with one(s) of each other via a second bus 252. Examples of the second bus 252 include an I2C bus, a PCI bus, a PCIe bus, and a SPI bus. Any other type of bus, such as a computing and/or electrical bus, is contemplated.

The second data interface(s) 232 of the illustrated example can be configured to transmit data and/or receive data. For example, the second data interface(s) 232 can be a bus. In some embodiments, the second data interface(s) 232 can be one or more communication interfaces.

In some embodiments, the second data interface(s) 232 can be configured to transmit data to and/or receive data from the fundus camera 202 via the first data interface(s) 212. In some embodiments, the second data interface(s) 232 can be configured to transmit data to and/or receive data from the electronic device(s) 136.

In some embodiments, the second data interface(s) 232 can be configured to transmit data to and/or receive data from the external electronic device(s) 140 and/or, more generally, the network 138. For example, the second data interface(s) 232 can implement one or more wireline and/or one or more wireless receivers to facilitate data transfer with the network 138. Examples of wireline receivers include Ethernet interfaces and optical interfaces. Examples of wireless receivers include Wireless Fidelity (Wi-Fi) receivers, cellular modems, and satellite receivers (e.g., beyond-line-of-site (BLOS) satellite receivers, line-of-site (LOS) satellite receivers, etc.).

The autofocus lens/sensor module 234 of the illustrated example can be configured to transmit data to and/or receive data from the autofocus lens 115 and/or the autofocus sensor 117 of FIG. 1. For example, the autofocus lens/sensor module 234 can transmit commands, directions, and/or instructions to the autofocus lens 115 to cause the autofocus lens 115 to move in one or more directions. In such an example, the autofocus lens/sensor module 234 can output commands to actuate one or more actuators associated with the autofocus lens 115 to move the autofocus lens 115 towards or away from the subject's eye 106 along the x-direction of the camera-based coordinate system 132.

In some embodiments, the autofocus lens/sensor module 234 can transmit data to the autofocus sensor 117. For example, the autofocus lens/sensor module 234 can output commands to cause the autofocus sensor 117 to emit infrared light towards the subject's eye 106.

In some embodiments, the autofocus lens/sensor module 234 can receive data from the autofocus sensor 117. For example, the autofocus lens/sensor module 234 can receive digital data from the autofocus sensor 117. In such an example, the digital data can be representative of a digital image. Alternatively, the autofocus lens/sensor module 234 can provide the digital data to the image processing module 246 to convert the digital data into a digital image (e.g., an autofocus image).

The autofocus determination module 236 of the illustrated example can be configured to execute one or more autofocus functions to determine a diopter value that can be used to configure the autofocus lens 204. In some embodiments, the autofocus determination module 236 can be configured to iteratively and/or sequentially execute the same autofocus function to determine a plurality of respective diopter values and from which an optimal diopter value can be determined. In some embodiments, the autofocus determination module 236 can be configured to execute a plurality of different autofocus functions in sequence to determine a plurality of respective diopter values and from which an optimal diopter value can be determined.

In some embodiments, the autofocus determination module 236 can execute an autofocus function using a plurality of autofocus images, and each of which is captured at a position along the x-axis of the camera-based coordinate system 132 of FIG. 1. The autofocus determination module 236 can process each of the autofocus images into a focus value using the autofocus function. The autofocus determination module 236 can generate a focus curve using the focus values. The autofocus determination module 236 can identify a diopter value that corresponds to the maximum focus value of the focus curve.

The autofocus determination module 236 can process a plurality of diopter values (each of which generated by executing an autofocus function) into an autofocus parameter, which can be used to configure the autofocus lens 204. For example, the autofocus parameter can include a diopter value, which can be determined from a mean, median, mode, or other statistical processing of a plurality of diopter values determined from executing a plurality of autofocus functions.

By way of example, the autofocus lens/sensor module 234 can effectuate an autofocus function by causing the autofocus sensor 205 to capture a plurality of autofocus images along different respective positions of the x-axis. The autofocus lens/sensor module 234 can cause the autofocus sensor 205 to repeat this process a number of times, such as 5 for example. The autofocus determination module 236 can determine a diopter value for each of the processes. For example, the autofocus determination module 236 can determine diopter values of −2.5 D, −2.75 D, −2.7 D, −2.6 D, and-2.8 D for 5 executions of the autofocus function (e.g.,−2.5 D for a first autofocus function execution, −2.75 D for a second autofocus function execution, etc.). In such an example, the autofocus determination module 236 can determine to terminate autofocus function executions and select the median diopter value of −2.7 D, which can be used to configure the autofocus lens 204 to take the fundus image. Alternatively, the autofocus determination module 236 can select the mean diopter of −2.67 D from the diopter values of −2.5 D, −2.75 D, −2.6 D, and −2.8 D for configuration of the autofocus lens 204.

In some embodiments, the autofocus determination module 236 determines to stop executing autofocus functions based on the resulting diopter values. For example, the autofocus lens/sensor module 234 can cause the autofocus sensor 205 to generate 3 focus curves, each of which resulting in a diopter value of −2.5 D. In such an example, the autofocus determination module 236 can decide to terminate the autofocus function executions and select the median (or mean) diopter of −2.5 D. Beneficially, less rounds of autofocus functions are taken because each execution of the autofocus function returns the same value.

In some embodiments, the autofocus determination module 236 can be configured to execute spherical correction. Additionally or alternatively, the autofocus determination module 236 can be configured to execute cylindrical correction. For example, the autofocus determination module 236 can determine that cylindrical correction is needed if a curvature of the subject's eye 106 does not match field curvature. In such an example, if the subject has a large cylinder correction, the autofocus determination module 236 can take two or three images to cover the different diopter ranges, such as (Spherical Diopter+Cylinder Diopter), (just Spherical Diopter), and (Spherical Diopter−Cylinder Diopter), and then merge the images from different cylinder diopters and select the respective areas that are in focus and merge them into one fully focus image.

The pupil detection module 240 of the illustrated example can be configured to detect a presence of a pupil, such as the pupil 108, in an image. For example, the pupil detection module 240 can perform one or more image processing techniques to detect the pupil 108 in an image. In some embodiments, the pupil detection module 240 can perform at least one of an edge detection technique or a thresholding technique to detect the pupil 108 in the image.

In some embodiments, the pupil detection module 240 can perform blink detection by determining whether a subject is blinking, whether an iris and/or pupil is detected (e.g., by performing concentric ellipse detection), and/or whether an eyelid is detected (e.g., by performing line detection). In some embodiments, the pupil detection module 240 can be configured to determine whether the pupil 108 is sufficiently exposed to the fundus camera 202. For example, the pupil detection module 240 can perform one or more image processing techniques to determine whether the pupil 108 is covered by an eyelid of the eye 106 and/or to what degree the pupil 108 is covered.

The pupil measurement module 242 of the illustrated example can be configured to determine a measurement of a dimension associated with the pupil 108 and/or, more generally, the eye 106. Examples of the dimension include a depth (or thickness), a height, a width, and a length of the eye 106, or portion(s) thereof, such as the pupil 108. By way of example, the pupil measurement module 242 can determine a width of the pupil 108 by analyzing and/or evaluating an image in which the pupil 108 is detected. For example, the pupil measurement module 242 can determine the width of the pupil 108 by performing an image processing technique on an image that includes the pupil 108.

The image sensor command generator 244 of the illustrated example can be configured to generate and/or output a command to direct, instruct, trigger, and/or cause the fundus camera 202 to capture an image, such as a fundus image, of the target eye location 124 of FIG. 1. For example, the image sensor command generator 224 can trigger the fundus camera 202 to capture a fundus image using the autofocus parameter determined by the autofocus determination module 236. In such an example, the autofocus lens/sensor module 234 can configure the autofocus lens 204 using a diopter value indicated by the autofocus parameter.

In some embodiments, the image sensor command generator 244 can generate and/or output a command to the illumination source(s) 208 and/or the fundus image sensor 210 via the first and second data interface(s) 212, 232 such that the illumination source(s) 208 creates a flash and the fundus image sensor 210 substantially simultaneously captures an image of the target eye location 124. In some embodiments, the image sensor command generator 244 triggers the flash and captures, after determining that the pupil 108 is sufficiently open, or the pupil 108, and/or, more generally, the eye 106, is detected in a field of view of the fundus camera 202. Beneficially, by triggering the flash and capture after such determination(s), the image sensor command generator 244 can capture a fundus image with increased accuracy and efficiency compared to conventional fundus photography techniques.

In some embodiments, the image sensor command generator 244 can cause the illumination source(s) 208 to create a flash automatically and the fundus image sensor 210 to capture an image automatically and substantially simultaneously. For example, the image sensor command generator 244 can control the illumination source(s) 208 and/or the fundus image sensor 210 without user input. In such an example, the image sensor command generator 244 can invoke and/or trigger the illumination source(s) 208 in response to determining that the pupil 108 is sufficiently open, or the pupil 108, and/or, more generally, the eye 106, is detected in a field of view of the fundus camera 202. Beneficially, in some embodiments, the fundus image can be captured without user input by invoking and/or triggering the illumination source(s) 208 and the fundus image sensor 210 in response to such determination(s) and/or as soon as such determination(s) are made.

In some embodiments, the image sensor command generator 244 can be configured to perform opportunistic flashing. For example, the image sensor command generator 244 may determine and/or predict an optimal and/or otherwise improved time at which to trigger image capture by using (i) a dynamic pupil size trigger determined by the pupil measurement module 242 and/or (ii) a blink status trigger determined by the pupil detection module 240. In some such embodiments, the image sensor command generator 244 may determine to capture an image of the pupil 108 when the pupil 108 is detected and sufficiently open for fundus image capture.

The image processing module 246 of the illustrated example can be configured to generate and/or render an image based on image data. For example, the image processing module 246 can be configured to receive image data from the fundus camera 114 and render the image data into images. In another example, the image processing module 246 can be configured to receive image data from the fundus image sensor 210 and render the image data into one or more images, such as a fundus image.

In some embodiments, the image processing module 246 can be configured to generate and/or render an autofocus image based on output from an autofocus sensor, such as the autofocus sensor 117 of FIG. 1 and/or the autofocus sensor 205 of FIG. 2. For example, the image processing module 246 can receive, via the data interfaces 212, 232, output from a CCD and/or infrared detector and convert the output into a digital image for processing by the autofocus determination module 236.

The user interface module 248 of the illustrated example can be configured to generate, instantiate, and/or launch a user interface that can be presented to a human user of the fundus imaging system 200. In some embodiments, the user interface module 248 can display an image, such as a fundus image, on the user interface. In some embodiments, the user interface module 248 can output feedback, such as a display of an alert and/or outputting of specific instructions to the user and/or patient. Examples of the alert include an indication that the subject is closing their eye 106 such that the pupil 108 is covered and the pupil 108 is not sufficiently large to effectuate fundus image capture.

In some embodiments, the user interface module 248 can generate, output, and/or provide feedback indicative of the alert. Examples of feedback that may be provided include audible feedback, haptic and/or tactile (e.g., vibrotactile) feedback, visual feedback, and/or any combination(s) thereof.

In some embodiments, the user interface module 248 can generate, output, and/or provide audible feedback to a patient and/or a fundus camera user. For example, the user interface module 248 can output audio feedback, such as an audible alert, via one or more speakers. By way of example, the audible alert can be a human voice based message such as “Please open your eyes wider” after determining that the pupil 108 is covered and/or not sufficiently opened. By way of yet another example, the audible alert can be a human voice based message such as “Please blink” after determining an autofocus parameter for configuring the autofocus lens 204.

In some embodiments, the user interface module 248 can generate, output, and/or provide haptic and/or tactile feedback to a patient and/or a user. For example, the user interface module 248 can output a vibration or any other vibrotactile feedback to a haptic sensor that may be embedded and/or included in the eye goggles of the fundus imaging system 102, the chin rest of the fundus imaging system 102, grip(s) of the fundus imaging system 102, and/or a combination(s) thereof. For example, the haptic sensor may output the vibration to cause the patient to make an adjustment, such as increasing an eye opening of the eye 106. Examples of a haptic sensor include an eccentric rotating mass vibration (ERMV) actuator (e.g., an ERMV motor), a linear resonant actuator (LRA), and a piezoelectric actuator (e.g., a piezo-haptic sensor).

In some embodiments, the user interface module 248 can generate, output, and/or provide visual feedback to a patient and/or a user. For example, the user interface module 248 can output a real-time visual (e.g., within 1 second, 500 ms, 100 ms, 10 ms, etc., of real-time) for the user that may show pupil dimensions. In some embodiments, the real-time visual may include one or more slider bars. Examples of slider bars include a slider bar for pupil dimension and a slider bar for blink status.

The datastore 250 of the illustrated example can be configured to record and/or store data. Examples of recorded/stored data include an image (e.g., a fundus image, an infrared image, an autofocus image), autofocus parameters (e.g., diopter values), sequential sequence(s) of image(s), alert(s), notification(s), video, and eye and/or pupil measurements. Any other type of recorded/stored data is contemplated. For example, the fundus image sensor 210, and/or, more generally, the fundus camera 202, may capture raw video and store the captured raw video in the datastore 250.

In some embodiments, the datastore 250 can be implemented by any technology for storing data. For example, the datastore 250 can be implemented by a volatile memory (e.g., a Synchronous Dynamic Random Access Memory (SDRAM), a Dynamic Random Access Memory (DRAM), a RAMBUS Dynamic Random Access Memory (RDRAM), etc.) and/or a non-volatile memory (e.g., flash memory). The datastore 250 may additionally or alternatively be implemented by one or more double data rate (DDR) memories, such as DDR, DDR2, DDR3, DDR4, DDR5, mobile DDR (mDDR), etc. The datastore 250 may additionally or alternatively be implemented by one or more mass storage devices such as hard disk drive(s) (HDD(s)), compact disk (CD) drive(s), digital versatile disk (DVD) drive(s), solid-state disk (SSD) drive(s), etc. While in the illustrated example the datastore 250 is illustrated as a single datastore, the datastore 250 may be implemented by any number and/or type(s) of datastore. Furthermore, the data stored in the datastore 250 may be in any data format. Examples of data formats include a flat file, binary data, comma delimited data, tab delimited data, image data, and structured query language (SQL) structures.

In some embodiments, the datastore 250 may be implemented by a database system, such as one or more databases. The term “database” as used herein means an organized body of related data, regardless of the manner in which the data or the organized body thereof is represented. For example, the organized body of related data may be in the form of one or more of a table, a log, a map, a grid, a packet, a datagram, a frame, a file, an e-mail, a message, a document, a report, a list or in any other form.

While an example implementation of the fundus camera 114, the fundus image computing system 130, and/or, more generally, the fundus imaging system 102 of FIG. 1, is depicted in FIG. 2, other implementations are contemplated. For example, one or more blocks, components, functions, etc., of the fundus camera 202, the fundus image computing system 230, and/or, more generally, the fundus imaging system 200, may be combined or divided in any other way. The fundus camera 202, the fundus image computing system 230, and/or, more generally, the fundus imaging system 200 of the illustrated example may be implemented by hardware alone, or by a combination of hardware, software, and/or firmware. For example, the fundus camera 202, the fundus image computing system 230, and/or, more generally, the fundus imaging system 200, may be implemented by one or more analog or digital circuits (e.g., comparators, operational amplifiers, etc.), one or more hardware-implemented state machines, one or more programmable processors (e.g., central processing units (CPUs), digital signal processors (DSPs), field programmable gate arrays (FPGAs), graphics processing units (GPUs), etc.), one or more network interfaces (e.g., network interface circuitry, network interface cards (NICs), smart NICs, etc.), one or more application specific integrated circuits (ASICs), one or more memories (e.g., non-volatile memory, volatile memory, etc.), one or more mass storage disks or devices (e.g., HDDs, SSD drives, etc.), etc., and/or any combination(s) thereof.

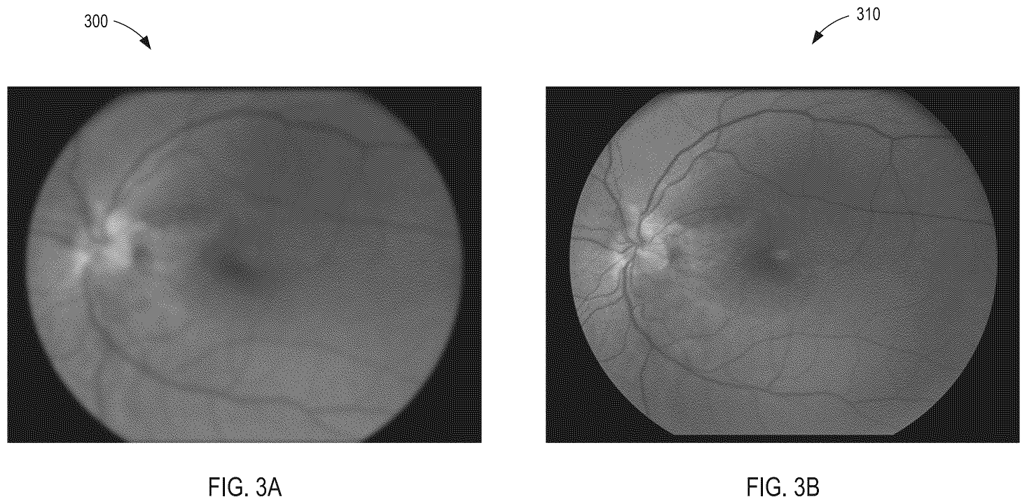

FIG. 3A is a fundus image 300. As shown in the illustrated example, the fundus image 300 is blurry and is not usable for clinical treatment of a human subject. The fundus image 300 may be captured using a fundus camera using an autofocus parameter determined from an execution of a single autofocus function.

FIG. 3B is a fundus image 310 captured with improved autofocus. As shown in the illustrated example, the fundus image 310 has improved sharpness and/or contrast with respect to the fundus image 300 of FIG. 3A. The fundus image 310 may be captured using the fundus camera 114 of FIG. 1 and/or, more generally, the fundus imaging system 102 of FIG. 1, and/or the fundus camera 202 of FIG. 2 and/or, more generally, the fundus image computing system 230 of FIG. 2.

By way of example, the fundus camera 114 may align the image path 126 of the fundus camera 114 to the pupil 108 of FIG. 1. The fundus camera 114 may iteratively execute an autofocus function to determine a plurality of diopter values. The fundus camera 114 may determine, using the plurality of diopter values, an autofocus parameter to adjust the focus of the autofocus lens 115 of FIG. 1. The fundus camera 114 can adjust the autofocus lens 115 in accordance with the autofocus parameter. The fundus camera 114 can capture, after adjusting the focus of the autofocus lens 115, the fundus image 310 of FIG. 3B.

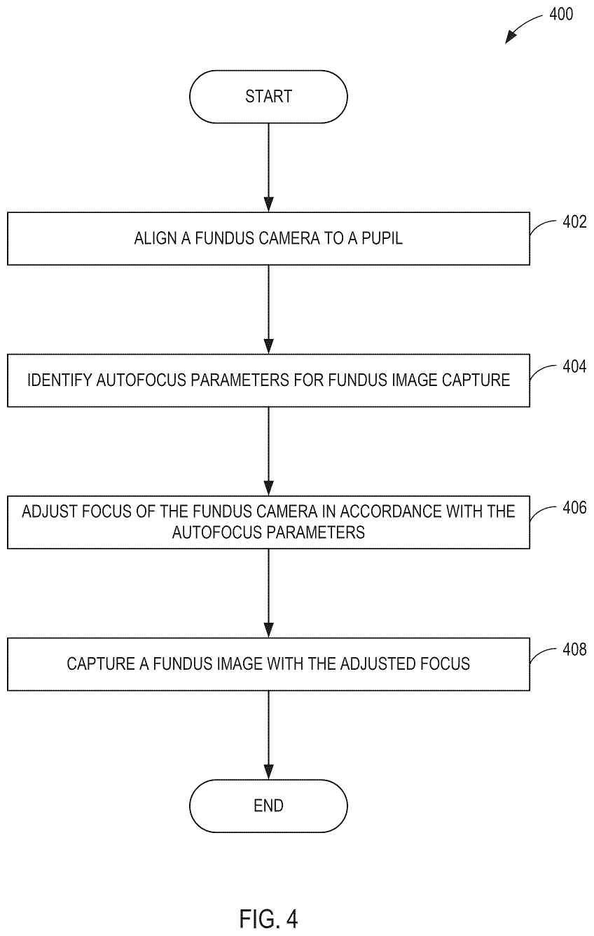

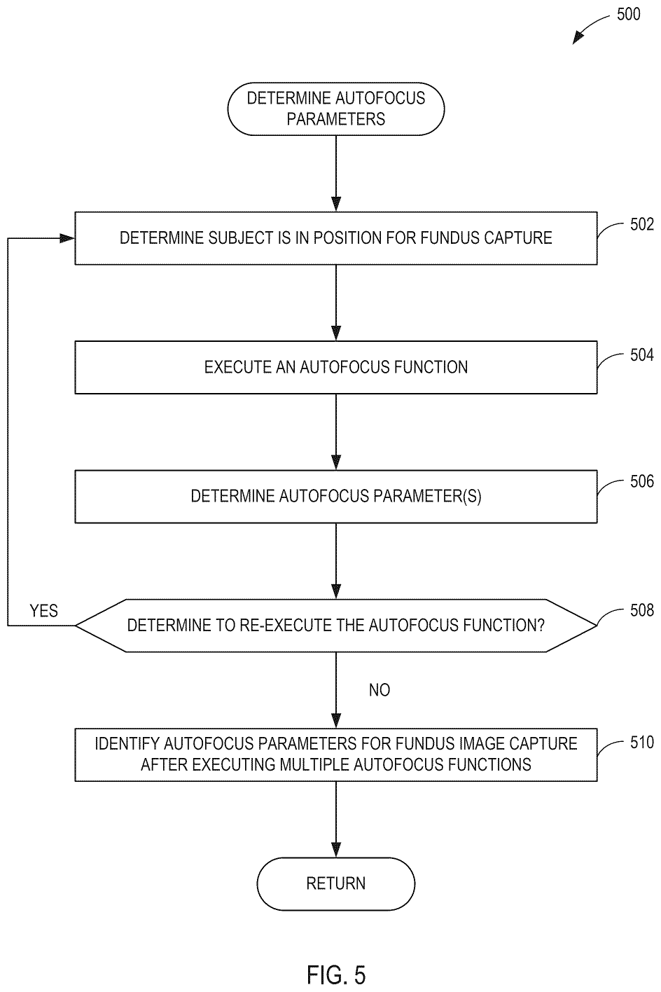

FIGS. 4 and 5 are flowcharts representative of example processes to be performed and/or example machine-readable instructions that may be executed by processor circuitry to implement a fundus imaging system, such as the fundus imaging system 102 of FIG. 1 and/or the fundus imaging system 200 of FIG. 2, or portion(s) thereof. Although a flowchart may be discussed in connection with one of the fundus imaging systems 102, 200 of FIGS. 1-2, the flowchart may also be applicable to other one(s) of the fundus imaging systems 102, 200 of FIGS. 1-2. Additionally or alternatively, block(s) of one(s) of the flowcharts of FIGS. 4 and/or 5 may be representative of state(s) of one or more hardware-implemented state machines, algorithm(s) that may be implemented by hardware alone such as an ASIC, etc., and/or any combination(s) thereof.

FIG. 4 is a flowchart 400 representative of an example process that may be performed and/or example machine-readable instructions that may be executed by processor circuitry to implement the fundus imaging system 102, 200 of FIGS. 1 and/or 2 to capture a fundus image of a retina of a subject using autofocus The flowchart 400 of FIG. 4 begins at block 402, at which the fundus imaging system 102, 200 of FIGS. 1 and/or 2 may align a fundus camera to a pupil. For example, the pupil detection module 240 can control the moveable platform 134 to align the imaging path 126 of the fundus camera 114 to the pupil 108 of the subject's eye 106.

At block 404, the fundus imaging system 102, 200 may identify autofocus parameters for fundus image capture. For example, the autofocus determination module 236 may identify a diopter value from a plurality of diopter values or determine the diopter value from the plurality of diopter values. In such an example, each of the plurality of diopter values may be determined from an autofocus function. The autofocus determination module 236 may determine the diopter value from the plurality of diopter values by determining the diopter value as a mean, a median, etc., of ones of the plurality of diopter values. Additionally or alternatively, the autofocus parameters may include specific quality numbers for the sharpness estimator for the autofocus function and/or focus fit quality estimates. The quality numbers for the sharpness estimator and/or the focus fit quality estimates may be collectively referred to as “quality measure values”.

At block 406, the fundus imaging system 102, 200 may adjust focus of the fundus camera in accordance with the autofocus parameters. For example, the autofocus lens/sensor module 234 may configure the autofocus lens 204 in accordance with the diopter value and/or, more generally, the autofocus parameters.

At block 408, the fundus imaging system 102, 200 may capture a fundus image with the adjusted focus. For example, the image sensor command generator 244 may instruct the fundus image sensor 210 to capture a fundus image of the target eye location 124 of FIG. 1 using the adjusted focus of the autofocus lens 204. In such an example, the fundus image can be stored in the datastore 250. The fundus image can be stored for eye health evaluation. The eye health evaluation can be performed by a medical professional via the electronic device(s) 136. Examples of a medical professional include a medical doctor (e.g., an ophthalmologist), an optometrist, and an optician. After capturing a fundus image with the adjusted focus at block 408, the example flowchart 400 of FIG. 4 concludes.

FIG. 5 is a flowchart 500 representative of an example process that may be performed and/or example machine-readable instructions that may be executed by processor circuitry to implement the fundus imaging system 102, 200 of FIGS. 1 and/or 2 to determine autofocus parameters for capturing a fundus image of a retina of a subject using autofocus. In some embodiments, the flowchart 500 of FIG. 5 may be executed to implement block 404 of the flowchart 400 of FIG. 4.

The flowchart 500 of FIG. 5 begins at block 502, at which the fundus imaging system 102, 200 of FIGS. 1 and/or 2 may determine that a subject is in position for fundus image capture. For example, the pupil detection module 240 may determine that the pupil 108 is detected.

At block 504, the fundus imaging system 102, 200 may execute an autofocus function. For example, the autofocus lens/sensor module 234 may capture an autofocus image at respective positions in a range of positions along the x-direction of the camera-based coordinate system 132 of FIG. 1. In such an example, the autofocus determination module 236 may determine a focus value for each of the autofocus images. The autofocus determination module 236 may determine a focus curve using the focus values. The autofocus determination module 236 may determine a diopter value that corresponds to the maximum focus value of the focus curve.

At block 506, the fundus imaging system 102, 200 may determine autofocus parameter(s). For example, the autofocus determination module 236 can determine and/or output a diopter value, such as the diopter value measured at block 504. Additionally and/or alternatively, the autofocus determination module 236 can determine and/or output a diopter value that is a mean, median, or mode of one or more determined diopter values, which can include the diopter value measured at block 504.

At block 508, the fundus imaging system 102, 200 may determine to re-execute the autofocus function. For example, the autofocus determination module 236 may determine that only a single execution of the autofocus function has been completed and one or more subsequent executions are needed. In another example, the autofocus determination module 236 may determine that a plurality of autofocus functions have been executed and a sufficient number of diopter values have been determined and/or a sufficiently low spread of diopter values is reached. In such an example, the autofocus determination module 236 may determine that 3 executions of the autofocus function have been complete and resulted in 3 respective diopter values that are the same or within a threshold deviation of each other (e.g., each of the 3 diopter values are within +0.1 or −0.1 of each other).

If, at block 508, the fundus imaging system 102, 200 determines to re-execute the autofocus function, control returns to block 502 to re-execute the autofocus function. Otherwise, control proceeds to block 510.

At block 510, the fundus imaging system 102, 200 may identify autofocus parameters for fundus image capture after executing multiple autofocus functions. For example, the autofocus determination module 236 may determine a median (or mean) of a plurality of diopter values, each of which resulting from a respective execution of an autofocus function. In such an example, the autofocus determination module 236 may identify the median diopter value as at least one of the autofocus parameters, which may be used to configure (e.g., focus, re-focus) the autofocus lens 115 of FIG. 1 and/or the autofocus lens 204 of FIG. 2 for fundus image capture.

After identifying autofocus parameters for fundus image capture after executing multiple autofocus functions at block 510, the example flowchart 500 of FIG. 5 concludes. For example, the flowchart 500 of FIG. 5 may return to block 406 of FIG. 4 to adjust focus of the fundus camera in accordance with the autofocus parameters.

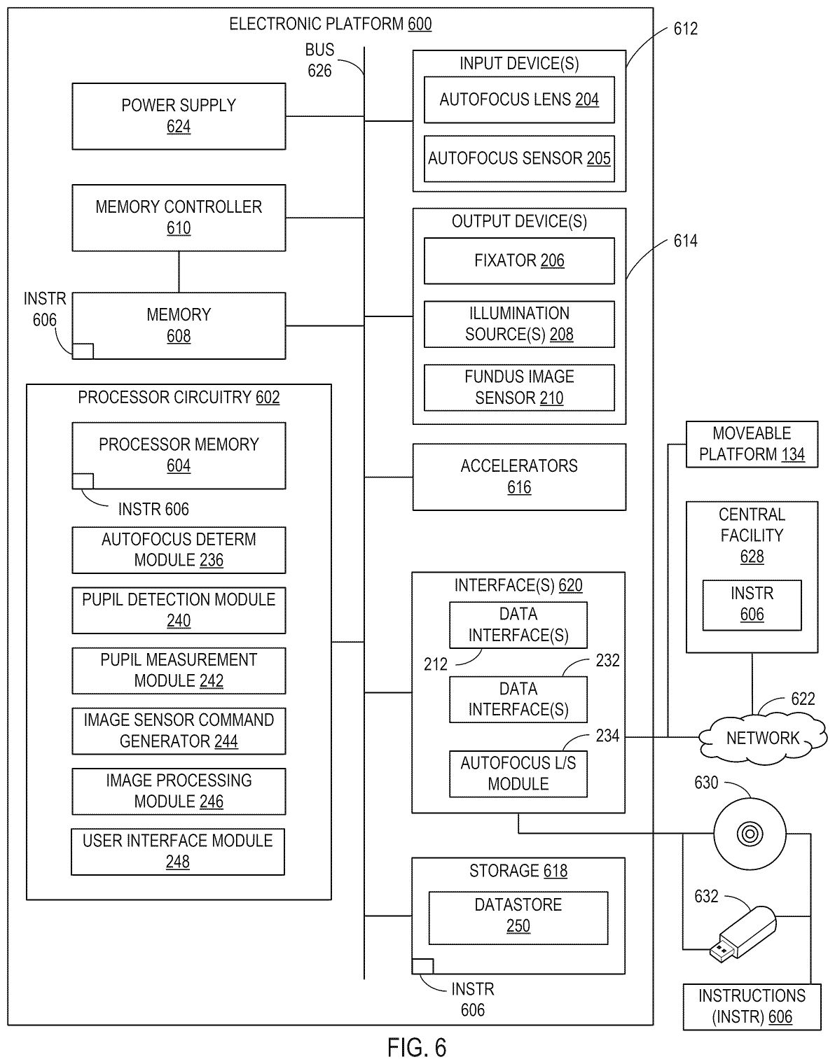

FIG. 6 is an example implementation of an electronic platform 600 structured to execute the machine-readable instructions of FIGS. 4 and/or 5 to implement a fundus imaging system, such as the fundus imaging system 102 of FIG. 1 and/or the fundus imaging system 200 of FIG. 2. In some embodiments, the electronic platform 600 can implement a portion of the fundus imaging system 102 of FIG. 1, such as the fundus camera 114 and/or the fundus image computing system 120 of FIG. 1. In some embodiments, the electronic platform 600 can implement a portion of the fundus imaging system 200 of FIG. 2, such as the fundus camera 202 and/or the fundus image computing system 230 of FIG. 2.

It should be appreciated that FIG. 6 is intended neither to be a description of necessary components for an electronic and/or computing device to operate as a fundus camera system, in accordance with the techniques described herein, nor a comprehensive depiction. The electronic platform 600 of this example may be an electronic device, such as an imaging system, an industrial computer, a server (e.g., a computer server, a blade server, a rack-mounted server, etc.), a workstation, or any other type of computing and/or electronic device. For example, the electronic platform 600 may be an electronic device coupled to and/or configured to be coupled to a fundus camera to implement a fundus imaging system.

The electronic platform 600 of the illustrated example includes processor circuitry 602, which may be implemented by one or more programmable processors, one or more hardware-implemented state machines, one or more ASICs, etc., and/or any combination(s) thereof. For example, the one or more programmable processors may include one or more CPUs, one or more DSPs, one or more FPGAs, one or more GPUs, etc., and/or any combination(s) thereof. The processor circuitry 602 includes processor memory 604, which may be volatile memory, such as random-access memory (RAM) of any type. The processor circuitry 602 of this example implements the autofocus determination module 236 (identified by AUTOFOCUS DETERM MODULE), the pupil detection module 240, the pupil measurement module 242, the image sensor command generator 244, the image processing module 246, and the user interface module 248 of FIG. 2.

The processor circuitry 602 may execute machine-readable instructions 606 (identified by INSTRUCTIONS), which are stored in the processor memory 604, to implement at least one of the autofocus determination module 236, the pupil detection module 240, the pupil measurement module 242, the image sensor command generator 244, the image processing module 246, or the user interface module 248 of FIG. 2. The machine-readable instructions 606 may include data representative of computer-executable and/or machine-executable instructions implementing techniques that operate according to the techniques described herein. For example, the machine-readable instructions 606 may include data (e.g., code, embedded software (e.g., firmware), software, etc.) representative of the flowcharts of FIGS. 4 and/or 5, or portion(s) thereof.

The electronic platform 600 includes memory 608, which may include the instructions 606. The memory 608 of this example may be controlled by a memory controller 610. For example, the memory controller 610 may control reads, writes, and/or, more generally, access(es) to the memory 608 by other component(s) of the electronic platform 600. The memory 608 of this example may be implemented by volatile memory, non-volatile memory, etc., and/or any combination(s) thereof. For example, the volatile memory may include static random-access memory (SRAM), dynamic random-access memory (DRAM), cache memory (e.g., Level 1 (L1) cache memory, Level 2 (L2) cache memory, Level 3 (L3) cache memory, etc.), etc., and/or any combination(s) thereof. In some examples, the non-volatile memory may include Flash memory, electrically erasable programmable read-only memory (EEPROM), magnetoresistive random-access memory (MRAM), ferroelectric random-access memory (FeRAM, F-RAM, or FRAM), etc., and/or any combination(s) thereof.

The electronic platform 600 includes input device(s) 612 to enable data and/or commands to be entered into the processor circuitry 602. For example, the input device(s) 612 may include an audio sensor or any other type of sensor, a camera (e.g., a still camera, a video camera, etc.), a keyboard, a microphone, a mouse, a touchscreen, a voice recognition system, etc., and/or any combination(s) thereof. In this example, the input device(s) 612 implement(s) the autofocus lens 204 and the autofocus sensor 205 of FIG. 2.

The electronic platform 600 includes output device(s) 614 to convey, display, and/or present information to a user (e.g., a human user, a machine user, etc.). For example, the output device(s) 614 may include one or more display devices, speakers, etc. The one or more display devices may include an augmented reality (AR) and/or virtual reality (VR) display, a liquid crystal display (LCD), a light-emitting diode (LED) display, an organic light-emitting diode (OLED) display, a quantum dot (QLED) display, a thin-film transistor (TFT) LCD, a touchscreen, etc., and/or any combination(s) thereof. The output device(s) 614 can be used, among other things, to generate, launch, and/or present a user interface. For example, the user interface may be generated and/or implemented by the output device(s) 614 for visual presentation of output and speakers or other sound generating devices for audible presentation of output. In this example, the output device(s) 614 implement(s) the fixator 206, the illumination source(s) 208, and the fundus image sensor 210 of FIG. 2. Additionally or alternatively, the user interface module 248 may be implemented by the output device(s) 614.

The electronic platform 600 includes accelerators 616, which are hardware devices to which the processor circuitry 602 may offload compute tasks to accelerate their processing. For example, the accelerators 616 may include artificial intelligence/machine-learning (AI/ML) processors, ASICs, FPGAs, GPUs, neural network (NN) processors, systems-on-chip (SoCs), vision processing units (VPUs), etc., and/or any combination(s) thereof. In some examples, one or more of the autofocus determination module 236, the pupil detection module 240, the pupil measurement module 242, the image sensor command generator 244, the image processing module 246, and/or the user interface module 248 may be implemented by one(s) of the accelerators 616 instead of the processor circuitry 602. In some examples, the autofocus determination module 236, the pupil detection module 240, the pupil measurement module 242, the image sensor command generator 244, the image processing module 246, and/or the user interface module 248 may be executed concurrently (e.g., in parallel, substantially in parallel, etc.) by the processor circuitry 602 and the accelerators 616. For example, the processor circuitry 602 and one(s) of the accelerators 616 may execute in parallel function(s) corresponding to the cardiac cycle analysis module 238.

The electronic platform 600 includes storage 618 to record and/or control access to data, such as the machine-readable instructions 606. In this example, the storage 618 implements the datastore 250 of FIG. 2. The storage 618 may be implemented by one or more mass storage disks or devices, such as HDDs, SSDs, etc., and/or any combination(s) thereof.

The electronic platform 600 includes interface(s) 620 to effectuate exchange of data with external devices (e.g., computing and/or electronic devices of any kind) via a network 622. In this example, the interface(s) 620 implement the first data interface(s) 212, the second data interface(s) 232, and the autofocus lens/sensor module 234 (identified by AUTOFOCUS L/S MODULE) of FIG. 2. Alternatively, the autofocus lens/sensor module 234 may be implemented at least in part by the processor circuitry 602.

The interface(s) 620 of the illustrated example may be implemented by an interface device, such as network interface circuitry (e.g., a NIC, a smart NIC, etc.), a gateway, a router, a switch, etc., and/or any combination(s) thereof. The interface(s) 620 may implement any type of communication interface, such as BLUETOOTH®, a cellular telephone system (e.g., a 4G LTE interface, a 5G interface, a future generation 6G interface, etc.), an Ethernet interface, a near-field communication (NFC) interface, an optical disc interface (e.g., a Blu-ray disc drive, a Compact Disk (CD) drive, a Digital Versatile Disk (DVD) drive, etc.), an optical fiber interface, a satellite interface (e.g., a BLOS satellite interface, a LOS satellite interface, etc.), a Universal Serial Bus (USB) interface (e.g., USB Type-A, USB Type-B, USB TYPE-C™ or USB-CT™, etc.), etc., and/or any combination(s) thereof. Additionally or alternatively, the interface(s) 620 may implement any other type of interface such as an I2C interface, a PCI interface, a PCIe interface, a SPI interface, and/or the like. For example, the interface(s) 620 may facilitate communication with the autofocus lens 115, the autofocus sensor 117, and/or the moveable platform 134 via any one(s) of the aforementioned interfaces.

The electronic platform 600 includes a power supply 624 to store energy and provide power to components of the electronic platform 600. The power supply 624 may be implemented by a power converter, such as an alternating current-to-direct-current (AC/DC) power converter, a direct current-to-direct current (DC/DC) power converter, etc., and/or any combination(s) thereof. For example, the power supply 624 may be powered by an external power source, such as an alternating current (AC) power source (e.g., an electrical grid), a direct current (DC) power source (e.g., a battery, a battery backup system, etc.), etc., and the power supply 624 may convert the AC input or the DC input into a suitable voltage for use by the electronic platform 600. In some examples, the power supply 624 may be a limited duration power source, such as a battery (e.g., a rechargeable battery such as a lithium-ion battery).

Component(s) of the electronic platform 600 may be in communication with one(s) of each other via a bus 626. For example, the bus 626 may be any type of computing and/or electrical bus, such as an I2C bus, a PCI bus, a PCIe bus, a SPI bus, and/or the like. In some embodiments, the bus 626 of FIG. 6 can implement and/or correspond to the first bus 222 and/or the second bus 252 of FIG. 2.

The network 622 may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more wireless local area networks (WLANs), etc., and/or any combination(s) thereof. For example, the network 622 may be the Internet, but any other type of private and/or public network is contemplated.

The network 622 of the illustrated example facilitates communication between the interface(s) 620 and a central facility 628. The central facility 628 in this example may be an entity associated with one or more servers, such as one or more physical hardware servers and/or virtualizations of the one or more physical hardware servers. For example, the central facility 628 may be implemented by a public cloud provider, a private cloud provider, etc., and/or any combination(s) thereof. In this example, the central facility 628 may compile, generate, update, etc., the machine-readable instructions 606 and store the machine-readable instructions 606 for access (e.g., download) via the network 622. For example, the electronic platform 600 may transmit a request, via the interface(s) 620, to the central facility 628 for the machine-readable instructions 606 and receive the machine-readable instructions 606 from the central facility 628 via the network 622 in response to the request.

Additionally or alternatively, the interface(s) 620 may receive the machine-readable instructions 606 via non-transitory machine-readable storage media, such as an optical disc 630 (e.g., a Blu-ray disc, a CD, a DVD, etc.) or any other type of removable non-transitory machine-readable storage media such as a USB drive 632. For example, the optical disc 630 and/or the USB drive 632 may store the machine-readable instructions 606 thereon and provide the machine-readable instructions 606 to the electronic platform 600 via the interface(s) 620.

Techniques operating according to the principles described herein may be implemented in any suitable manner. The processing and decision blocks of the flowcharts above represent steps and acts that may be included in algorithms that carry out these various processes. Algorithms derived from these processes may be implemented as software integrated with and directing the operation of one or more single-or multi-purpose processors, may be implemented as functionally equivalent circuits such as a DSP circuit or an ASIC, or may be implemented in any other suitable manner. It should be appreciated that the flowcharts included herein do not depict the syntax or operation of any particular circuit or of any particular programming language or type of programming language. Rather, the flowcharts illustrate the functional information one skilled in the art may use to fabricate circuits or to implement computer software algorithms to perform the processing of a particular apparatus carrying out the types of techniques described herein. For example, the flowcharts, or portion(s) thereof, may be implemented by hardware alone (e.g., one or more analog or digital circuits, one or more hardware-implemented state machines, etc., and/or any combination(s) thereof) that is configured or structured to carry out the various processes of the flowcharts. In some examples, the flowcharts, or portion(s) thereof, may be implemented by machine-executable instructions (e.g., machine-readable instructions, computer-readable instructions, computer-executable instructions, etc.) that, when executed by one or more single-or multi-purpose processors, carry out the various processes of the flowcharts. It should also be appreciated that, unless otherwise indicated herein, the particular sequence of steps and/or acts described in each flowchart is merely illustrative of the algorithms that may be implemented and can be varied in implementations and embodiments of the principles described herein.

Accordingly, in some embodiments, the techniques described herein may be embodied in machine-executable instructions implemented as software, including as application software, system software, firmware, middleware, embedded code, or any other suitable type of computer code. Such machine-executable instructions may be generated, written, etc., using any of a number of suitable programming languages and/or programming or scripting tools, and also may be compiled as executable machine language code or intermediate code that is executed on a framework, virtual machine, or container.

When techniques described herein are embodied as machine-executable instructions, these machine-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc., that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement using the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (e.g., as a single unit or separate units), or some of these functional facilities may not be implemented.

Machine-executable instructions (e.g., processor-executable instructions) implementing the techniques described herein (when implemented as one or more functional facilities or in any other manner) may, in some embodiments, be encoded on one or more computer-readable media, machine-readable media, etc., to provide functionality to the media. Computer-readable media, machine-readable media, etc., include magnetic media such as a hard disk drive, optical media such as a CD or a DVD, a persistent or non-persistent solid-state memory (e.g., Flash memory, Magnetic RAM, etc.), or any other suitable storage media. Such a computer-readable medium, a machine-readable medium, etc., may be implemented in any suitable manner. As used herein, the terms “computer-readable media” (also called “computer-readable storage media”), “computer-readable medium” (also called “computer-readable storage medium”), “machine-readable media” (also called “machine-readable storage media”), and “machine-readable medium” (also called “machine-readable storage medium”) refer to tangible storage media. Tangible storage media are non-transitory and have at least one physical, structural component. In a “computer-readable medium” and “machine-readable medium” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium, a machine-readable medium, etc., may be altered during a recording process.

Further, some techniques described above comprise acts of storing information (e.g., data and/or instructions) in certain ways for use by these techniques. In some implementations of these techniques—such as implementations where the techniques are implemented as machine-executable instructions—the information may be encoded on a computer-readable storage media. Where specific structures are described herein as advantageous formats in which to store this information, these structures may be used to impart a physical organization of the information when encoded on the storage medium.

These advantageous structures may then provide functionality to the storage medium by affecting operations of one or more processors interacting with the information; for example, by increasing the efficiency of computer operations performed by the processor(s).

In some, but not all, implementations in which the techniques may be embodied as machine-executable instructions, these instructions may be executed on one or more suitable computing device(s) and/or electronic device(s) operating in any suitable computer and/or electronic system, or one or more computing devices (or one or more processors of one or more computing devices) and/or one or more electronic devices (or one or more processors of one or more electronic devices) may be programmed to execute the machine-executable instructions. A computing device, electronic device, or processor (e.g., processor circuitry) may be programmed to execute instructions when the instructions are stored in a manner accessible to the computing device, electronic device, or processor, such as in a data store (e.g., an on-chip cache or instruction register, a computer-readable storage medium and/or a machine-readable storage medium accessible via a bus, a computer-readable storage medium and/or a machine-readable storage medium accessible via one or more networks and accessible by the device/processor, etc.). Functional facilities comprising these machine-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more FPGAs for carrying out the techniques described herein, or any other suitable system.

Embodiments have been described where the techniques are implemented in circuitry and/or machine-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The phrase “and/or,” as used herein in the specification and in the claims, should be understood to mean “either or both,” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The indefinite articles “a” and “an,” as used herein in the specification and in the claims, unless clearly indicated to the contrary, should be understood to mean “at least one.” As used herein in the specification and in the claims, the phrase, “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A,, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The word “exemplary” is used herein to mean serving as an example, instance, or illustration. Any embodiment, implementation, process, feature, etc., described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

Various aspects are described in this disclosure, which include, but are not limited to, the following aspects:
1. A method for fundus imaging, comprising: aligning an image path of a fundus camera to an eye pupil of a subject; iteratively executing an autofocus function to determine a plurality of first diopter values; adjusting a focus of a lens of the fundus camera in accordance with a second diopter value, the second diopter value identified from the plurality of first diopter values or determined from the plurality of first diopter values; and capturing, after adjusting the focus of the lens in accordance with the second diopter value, a fundus image of a retina of the subject.
2. The method of aspect 1, wherein aligning the image path of the fundus camera with the eye pupil comprises controlling at least one actuator to move the fundus camera in one or more directions.
3. The method of any one of aspects 1-2, further comprising capturing image data of the eye pupil of the subject using an infrared sensor.
4. The method of any one of aspects 1-3, further comprising determining whether the subject blinked, and wherein capturing the fundus image comprises capturing the fundus image after determining that an eyelid of the subject reopened after blinking.
5. The method of any one of aspects 1-4, further comprising storing the fundus image for eye health evaluation.
6. The method of any one of aspects 1-5, wherein iteratively executing the autofocus function comprises: executing a first instance of the autofocus function by processing first image data captured along the image path to determine a third diopter value of the plurality of first diopter values, the third diopter value associated with the focus of the lens of the fundus camera; and executing a second instance of the autofocus function by processing second image data captured along the image path to determine a fourth diopter value of the plurality of first diopter values, the fourth diopter value associated with the focus of the lens of the fundus camera.
7. The method of any one of aspects 1-6, wherein iteratively executing the autofocus function comprises executing a first instance of the autofocus function, and executing the first instance of the autofocus function comprises: capturing a plurality of autofocus images by capturing an autofocus image at different fundus camera positions in a range of different camera positions; converting the plurality of autofocus images into frequency data; identifying which one of the plurality of autofocus images has a greatest number of high frequency components of the frequency data; and identifying a candidate diopter value that corresponds to one of the fundus camera positions at which the identified autofocus image was captured.
8. The method of any one of aspects 1-7, wherein the plurality of autofocus images is a first plurality of autofocus images, the frequency data is first frequency data, and iteratively executing the autofocus function comprises executing, after the first instance of the autofocus function, a second instance of the autofocus function comprising: capturing a second plurality of autofocus images by capturing an autofocus image at the different fundus camera positions in the range of different camera positions; converting the second plurality of the autofocus images into second frequency data; identifying which one of the second plurality of autofocus images has a greatest number of high frequency components of the frequency data; and identifying a second candidate diopter value that corresponds to one of the fundus camera positions at which the identified autofocus image was captured.
9. The method of any one of aspects 1-8, further comprising: calculating an autofocus parameter based on the first candidate diopter value and the second candidate diopter value; and determining the second diopter value based on the autofocus parameter.
10. The method of any one of aspects 1-9, wherein the autofocus parameter is at least one of a median, mean, or mode of at least the first candidate diopter value and the second candidate diopter value.
11. An apparatus for fundus imaging, comprising: at least one memory storing processor-executable instructions; and at least one computer hardware processor configured to execute the processor-executable instructions to perform a method comprising: aligning an image path of a fundus camera to an eye pupil of a subject; iteratively executing an autofocus function to determine a plurality of first diopter values; adjusting a focus of a lens of the fundus camera in accordance with a second diopter value, the second diopter value identified from the plurality of first diopter values or determined from the plurality of first diopter values; and capturing, after adjusting the focus of the lens in accordance with the second diopter value, a fundus image of a retina of the subject.
12. The apparatus of aspect 11, wherein aligning the image path of the fundus camera with the eye pupil comprises controlling at least one actuator to move the fundus camera in one or more directions.
13. The apparatus of any one of aspects 11-12, the method further comprising capturing image data of the eye pupil of the subject using an infrared sensor.
14. The apparatus of any one of aspects 11-13, the method further comprising determining whether the subject blinked, and wherein capturing the fundus image comprises capturing the fundus image after determining that an eyelid of the subject reopened after blinking.
15. The apparatus of any one of aspects 11-14, the method further comprising storing the fundus image for eye health evaluation.
16. The apparatus of any one of aspects 11-15, wherein iteratively executing the autofocus function comprises: executing a first instance of the autofocus function by processing first image data captured along the image path to determine a third diopter value of the plurality of first diopter values, the third diopter value associated with the focus of the lens of the fundus camera; and executing a second instance of the autofocus function by processing second image data captured along the image path to determine a fourth diopter value of the plurality of first diopter values, the fourth diopter value associated with the focus of the lens of the fundus camera.
17. The apparatus of any one of aspects 11-16, wherein iteratively executing the autofocus function comprises executing a first instance of the autofocus function, and executing the first instance of the autofocus function comprises: capturing a plurality of autofocus images by capturing an autofocus image at different fundus camera positions in a range of different camera positions; converting the plurality of autofocus images into frequency data; identifying which one of the plurality of autofocus images has a greatest number of high frequency components of the frequency data; and identifying a candidate diopter value that corresponds to one of the fundus camera positions at which the identified autofocus image was captured.
18. The apparatus of any one of aspects 11-17, wherein the plurality of autofocus images is a first plurality of autofocus images, the frequency data is first frequency data, and iteratively executing the autofocus function comprises executing, after the first instance of the autofocus function, a second instance of the autofocus function comprising: capturing a second plurality of autofocus images by capturing an autofocus image at the different fundus camera positions in the range of different camera positions; converting the second plurality of the autofocus images into second frequency data; identifying which one of the second plurality of autofocus images has a greatest number of high frequency components of the frequency data; and identifying a second candidate diopter value that corresponds to one of the fundus camera positions at which the identified autofocus image was captured.
19. The apparatus of any one of aspects 11-18, the method further comprising: calculating an autofocus parameter based on the first candidate diopter value and the second candidate diopter value; and determining the second diopter value based on the autofocus parameter.
20. The apparatus of any one of aspects 11-19, wherein the autofocus parameter is at least one of a median, mean, or mode of at least the first candidate diopter value and the second candidate diopter value.
21. At least one non-transitory computer-readable storage medium comprising processor-executable instructions that, when executed by at least one computer hardware processor, cause the at least one computer hardware processor to perform a method for fundus imaging, the method comprising: aligning an image path of a fundus camera to an eye pupil of a subject; iteratively executing an autofocus function to determine a plurality of first diopter values; adjusting a focus of a lens of the fundus camera in accordance with a second diopter value, the second diopter value identified from the plurality of first diopter values or determined from the plurality of first diopter values; and capturing, after adjusting the focus of the lens in accordance with the second diopter value, a fundus image of a retina of the subject.
22. The at least one non-transitory computer-readable storage medium of aspect 21, wherein aligning the image path of the fundus camera with the eye pupil comprises controlling at least one actuator to move the fundus camera in one or more directions.
23. The at least one non-transitory computer-readable storage medium of any one of aspects 21-22, the method further comprising capturing image data of the eye pupil of the subject using an infrared sensor.
24. The at least one non-transitory computer-readable storage medium of any one of aspects 21-23, the method further comprising determining whether the subject blinked, and wherein capturing the fundus image comprises capturing the fundus image after determining that an eyelid of the subject reopened after blinking.
25. The at least one non-transitory computer-readable storage medium of any one of aspects 21-24, the method further comprising storing the fundus image for eye health evaluation.
26. The at least one non-transitory computer-readable storage medium of any one of aspects 21-25, wherein iteratively executing the autofocus function comprises: executing a first instance of the autofocus function by processing first image data captured along the image path to determine a third diopter value of the plurality of first diopter values, the third diopter value associated with the focus of the lens of the fundus camera; and executing a second instance of the autofocus function by processing second image data captured along the image path to determine a fourth diopter value of the plurality of first diopter values, the fourth diopter value associated with the focus of the lens of the fundus camera.
27. The at least one non-transitory computer-readable storage medium of any one of aspects 21-26, wherein iteratively executing the autofocus function comprises executing a first instance of the autofocus function, and executing the first instance of the autofocus function comprises: capturing a plurality of autofocus images by capturing an autofocus image at different fundus camera positions in a range of different camera positions; converting the plurality of autofocus images into frequency data; identifying which one of the plurality of autofocus images has a greatest number of high frequency components of the frequency data; and identifying a candidate diopter value that corresponds to one of the fundus camera positions at which the identified autofocus image was captured.
28. The at least one non-transitory computer-readable storage medium of any one of aspects 21-27, wherein the plurality of autofocus images is a first plurality of autofocus images, the frequency data is first frequency data, and iteratively executing the autofocus function comprises executing, after the first instance of the autofocus function, a second instance of the autofocus function comprising: capturing a second plurality of autofocus images by capturing an autofocus image at the different fundus camera positions in the range of different camera positions; converting the second plurality of the autofocus images into second frequency data; identifying which one of the second plurality of autofocus images has a greatest number of high frequency components of the frequency data; and identifying a second candidate diopter value that corresponds to one of the fundus camera positions at which the identified autofocus image was captured.
29. The at least one non-transitory computer-readable storage medium of any one of aspects 21-28, the method further comprising: calculating an autofocus parameter based on the first candidate diopter value and the second candidate diopter value; and determining the second diopter value based on the autofocus parameter.
30. The at least one non-transitory computer-readable storage medium of any one of aspects 21-29, wherein the autofocus parameter is at least one of a median, mean, or mode of at least the first candidate diopter value and the second candidate diopter value.
31. A fundus imaging system comprising: a fundus camera; at least one memory storing processor-executable instructions; and at least one computer hardware processor configured to execute the processor-executable instructions to perform a method for fundus imaging, the method comprising: aligning an image path of the fundus camera to an eye pupil of a subject; iteratively executing an autofocus function to determine a plurality of first diopter values; adjusting a focus of a lens of the fundus camera in accordance with a second diopter value, the second diopter value identified from the plurality of first diopter values or determined from the plurality of first diopter values; and capturing, after adjusting the focus of the lens in accordance with the second diopter value, a fundus image of a retina of the subject.
32. The fundus imaging system of aspect 31, further comprising at least one actuator, and wherein aligning the image path of the fundus camera with the eye pupil comprises controlling the at least one actuator to move the fundus camera in one or more directions.
33. The fundus imaging system of any one of aspects 31-32, wherein the fundus camera comprises an infrared sensor, and the method further comprising capturing image data of the eye pupil of the subject using the infrared sensor.
34. The fundus imaging system of any one of aspects 31-33, the method further comprising determining whether the subject blinked, and wherein capturing the fundus image comprises capturing the fundus image after determining that an eyelid of the subject reopened after blinking.
35. The fundus imaging system of any one of aspects 31-34, the method further comprising storing the fundus image for eye health evaluation.
36. The fundus imaging system of any one of aspects 31-35, wherein iteratively executing the autofocus function comprises: executing a first instance of the autofocus function by processing first image data captured along the image path to determine a third diopter value of the plurality of first diopter values, the third diopter value associated with the focus of the lens of the fundus camera; and executing a second instance of the autofocus function by processing second image data captured along the image path to determine a fourth diopter value of the plurality of first diopter values, the fourth diopter value associated with the focus of the lens of the fundus camera.
37. The fundus imaging system of any one of aspects 31-36, wherein iteratively executing the autofocus function comprises executing a first instance of the autofocus function, and executing the first instance of the autofocus function comprises: capturing a plurality of autofocus images by capturing an autofocus image at different fundus camera positions in a range of different camera positions; converting the plurality of autofocus images into frequency data; identifying which one of the plurality of autofocus images has a greatest number of high frequency components of the frequency data; and identifying a candidate diopter value that corresponds to one of the fundus camera positions at which the identified autofocus image was captured.
38. The fundus imaging system of any one of aspects 31-37, wherein the plurality of autofocus images is a first plurality of autofocus images, the frequency data is first frequency data, and iteratively executing the autofocus function comprises executing, after the first instance of the autofocus function, a second instance of the autofocus function comprising: capturing a second plurality of autofocus images by capturing an autofocus image at the different fundus camera positions in the range of different camera positions; converting the second plurality of the autofocus images into second frequency data; identifying which one of the second plurality of autofocus images has a greatest number of high frequency components of the frequency data; and identifying a second candidate diopter value that corresponds to one of the fundus camera positions at which the identified autofocus image was captured.
39. The fundus imaging system of any one of aspects 31-38, the method further comprising: calculating an autofocus parameter based on the first candidate diopter value and the second candidate diopter value; and determining the second diopter value based on the autofocus parameter.
40. The fundus imaging system of any one of aspects 31-39, wherein the autofocus parameter is at least one of a median, mean, or mode of at least the first candidate diopter value and the second candidate diopter value.

1. A method for fundus imaging, comprising:
aligning an image path of a fundus camera to an eye pupil of a subject;
iteratively executing an autofocus function to determine a plurality of first diopter values;
adjusting a focus of a lens of the fundus camera in accordance with a second diopter value, the second diopter value identified from the plurality of first diopter values or determined from the plurality of first diopter values; and
capturing, after adjusting the focus of the lens in accordance with the second diopter value, a fundus image of a retina of the subject.

2. The method of claim 1, wherein aligning the image path of the fundus camera with the eye pupil comprises controlling at least one actuator to move the fundus camera in one or more directions.

3. The method of claim 1, further comprising capturing image data of the eye pupil of the subject using an infrared sensor.

4. The method of claim 1, further comprising determining whether the subject blinked, and wherein capturing the fundus image comprises capturing the fundus image after determining that an eyelid of the subject reopened after blinking.

5. The method of claim 1, further comprising storing the fundus image for eye health evaluation.

6. The method of claim 1, wherein iteratively executing the autofocus function comprises:
executing a first instance of the autofocus function by processing first image data captured along the image path to determine a third diopter value of the plurality of first diopter values, the third diopter value associated with the focus of the lens of the fundus camera; and
executing a second instance of the autofocus function by processing second image data captured along the image path to determine a fourth diopter value of the plurality of first diopter values, the fourth diopter value associated with the focus of the lens of the fundus camera.

7. The method of claim 1, wherein iteratively executing the autofocus function comprises executing a first instance of the autofocus function, and executing the first instance of the autofocus function comprises:
capturing a plurality of autofocus images by capturing an autofocus image at different fundus camera positions in a range of different camera positions;
converting the plurality of autofocus images into frequency data;
identifying which one of the plurality of autofocus images has a greatest number of high frequency components of the frequency data; and
identifying a candidate diopter value that corresponds to one of the fundus camera positions at which the identified autofocus image was captured.

8. The method of claim 7, wherein the plurality of autofocus images is a first plurality of autofocus images, the frequency data is first frequency data, and iteratively executing the autofocus function comprises executing, after the first instance of the autofocus function, a second instance of the autofocus function comprising:
capturing a second plurality of autofocus images by capturing an autofocus image at the different fundus camera positions in the range of different camera positions;
converting the second plurality of the autofocus images into second frequency data;
identifying which one of the second plurality of autofocus images has a greatest number of high frequency components of the frequency data; and
identifying a second candidate diopter value that corresponds to one of the fundus camera positions at which the identified autofocus image was captured.

9. The method of claim 8, further comprising:
calculating an autofocus parameter based on the first candidate diopter value and the second candidate diopter value; and
determining the second diopter value based on the autofocus parameter.

10. The method of claim 9, wherein the autofocus parameter is at least one of a median, mean, or mode of at least the first candidate diopter value and the second candidate diopter value.

11. At least one non-transitory computer-readable storage medium comprising processor-executable instructions that, when executed by at least one computer hardware processor, cause the at least one computer hardware processor to perform a method for fundus imaging, the method comprising:
aligning an image path of a fundus camera to an eye pupil of a subject;
iteratively executing an autofocus function to determine a plurality of first diopter values;
adjusting a focus of a lens of the fundus camera in accordance with a second diopter value, the second diopter value identified from the plurality of first diopter values or determined from the plurality of first diopter values; and
capturing, after adjusting the focus of the lens in accordance with the second diopter value, a fundus image of a retina of the subject.

12. The at least one non-transitory computer-readable storage medium of claim 11, wherein iteratively executing the autofocus function comprises:
executing a first instance of the autofocus function by processing first image data captured along the image path to determine a third diopter value of the plurality of first diopter values, the third diopter value associated with the focus of the lens of the fundus camera; and
executing a second instance of the autofocus function by processing second image data captured along the image path to determine a fourth diopter value of the plurality of first diopter values, the fourth diopter value associated with the focus of the lens of the fundus camera.

13. The at least one non-transitory computer-readable storage medium of claim 11, wherein iteratively executing the autofocus function comprises executing a first instance of the autofocus function, and executing the first instance of the autofocus function comprises:
capturing a plurality of autofocus images by capturing an autofocus image at different fundus camera positions in a range of different camera positions;
converting the plurality of autofocus images into frequency data;
identifying which one of the plurality of autofocus images has a greatest number of high frequency components of the frequency data; and
identifying a candidate diopter value that corresponds to one of the fundus camera positions at which the identified autofocus image was captured.

14. The at least one non-transitory computer-readable storage medium of claim 13, wherein the plurality of autofocus images is a first plurality of autofocus images, the frequency data is first frequency data, and iteratively executing the autofocus function comprises executing, after the first instance of the autofocus function, a second instance of the autofocus function comprising:
capturing a second plurality of autofocus images by capturing an autofocus image at the different fundus camera positions in the range of different camera positions;
converting the second plurality of the autofocus images into second frequency data;
identifying which one of the second plurality of autofocus images has a greatest number of high frequency components of the frequency data; and
identifying a second candidate diopter value that corresponds to one of the fundus camera positions at which the identified autofocus image was captured.

15. The at least one non-transitory computer-readable storage medium of claim 14, the method further comprising:
calculating an autofocus parameter based on the first candidate diopter value and the second candidate diopter value; and
determining the second diopter value based on the autofocus parameter.

16. The at least one non-transitory computer-readable storage medium of claim 15, wherein the autofocus parameter is at least one of a median, mean, or mode of at least the first candidate diopter value and the second candidate diopter value.

17. A fundus imaging system comprising:
a fundus camera;
at least one memory storing processor-executable instructions; and
at least one computer hardware processor configured to execute the processor-executable instructions to perform a method for fundus imaging, the method comprising:
aligning an image path of the fundus camera to an eye pupil of a subject;
iteratively executing an autofocus function to determine a plurality of first diopter values;
adjusting a focus of a lens of the fundus camera in accordance with a second diopter value, the second diopter value identified from the plurality of first diopter values or determined from the plurality of first diopter values; and
capturing, after adjusting the focus of the lens in accordance with the second diopter value, a fundus image of a retina of the subject.

18. The fundus imaging system of claim 17, further comprising at least one actuator, and wherein aligning the image path of the fundus camera with the eye pupil comprises controlling the at least one actuator to move the fundus camera in one or more directions.

19. The fundus imaging system of claim 17, wherein the fundus camera comprises an infrared sensor, and the method further comprising capturing image data of the eye pupil of the subject using the infrared sensor.

20. The fundus imaging system of claim 17, wherein iteratively executing the autofocus function comprises:
executing a first instance of the autofocus function by processing first image data captured along the image path to determine a third diopter value of the plurality of first diopter values, the third diopter value associated with the focus of the lens of the fundus camera; and
executing a second instance of the autofocus function by processing second image data captured along the image path to determine a fourth diopter value of the plurality of first diopter values, the fourth diopter value associated with the focus of the lens of the fundus camera.
